(19) United States
(12) Patent Application Publication
(10) Pub. No.: US 2022/0299532 A1
(43) Pub. Date: Sep. 22, 2022

(54) CORRECTING PROTEIN MISFOLDING IN DIABETES

(71) Applicants: Sanford Burnham Prebys Medical Discovery Institute, La Jolla, CA (US); The Regents of the University of Michigan, Ann Arbor, MI (US)

(72) Inventors: Randal J. KAUFMAN, San Diego, CA (US); Peter ARVAN, Ann Arbor, MI (US)

(21) Appl. No.: 17/618,112

(22) PCT Filed: Jun. 9, 2020
(86) PCT No.: PCT/US2020/036835

§ 371 Date: Dec. 10, 2021

Related U.S. Application Data
(60) Provisional application No. 62/859,379, filed on Jun. 10, 2019.

(51) Int. Cl. G01N 33/74; G01N 33/68

(52) U.S. Cl. CPC G01N 33/74; G01N 33/6893; G01N 2333/62; G01N 2800/042; G01N 2800/50

(57) ABSTRACT

The present disclosure relates to proinsulin misfolding in beta cells as it relates to glucose intolerance associated with type 2 diabetes (T2D).

### CROSS-REFERENCE TO RELATED APPLICATIONS

This application claims the benefit of U.S. Provisional Patent Application No. 62/859,379 filed on Jun. 10, 2019. Priority is claimed pursuant to 35 U.S.C. § 119. The above noted patent application is incorporated by reference as if set forth fully herein.

### STATEMENT REGARDING FEDERALLY SPONSORED RESEARCH

This invention was made with government support under R24 DK110973 awarded by the National Institutes of Health (NIH). The government has certain rights in the invention.

### BACKGROUND OF THE DISCLOSURE

Type 2 diabetes mellitus (T2D) is a complex disease caused by multiple genetic and environmental factors with an overarching problem of insufficient insulin to meet the level of insulin resistance. Fundamental in the etiology of T2D are, among others, pancreatic β cell failure, including decreased β cell number, chronic ER stress and/or oxidative stress, and a loss of β cell identity. Pancreatic β cell failure may be heavily associated with insulin resistance, placing burden on the pancreatic β cells to increase insulin synthesis and secretion. For example, β cells may compensate for insulin resistance by increasing insulin production that may eventually overwhelm the ER capacity for efficient protein folding, thereby provoking β-cell ER stress. From a different perspective, it may be that chronic accumulation of misfolded proteins in the ER activates the unfolded protein response (UPR) through the ER stress transducers PERK, IRE1, and ATF6 to alleviate and adapt to the cellular stress, or hypersynthesis of unfolded peptides may promote excessive ER stress, which can eventually lead to loss of β cells or loss of activity of the β cells.

A mature and functional insulin is formed by proper folding of proinsulin peptide by disulfide bonds between A and B polypeptide chains (A7-B7 and A20-B19) and one in the A chain (A6-A11). Disulfide bond formation within the proinsulin peptide occurs during the early stages of protein folding via a catalytic reaction with ER oxidoreductin 1 (ERO1) that transfers oxidizing equivalents from O₂ to form disulfide bonds. Mutations within proinsulin that impact disulfide bond formation cause neonatal diabetes in humans and Akita mice, serving as a model of proinsulin misfolding-induced β cell failure. Similarly, in human, a mutation in INS allele causes proinsulin misfolding, leading to an autosomal-dominant form of diabetes known as Mutant INS-gene-induced Diabetes of Youth (MIDY). In the animal model of MIDY, it has been identified that peptides encoded by one mutant INS allele encoding proinsulin-C(A7)Y cannot form Cys(B7)-Cys(A7) disulfide bond, which leads to misfolding of the insulin protein. Yet, it is unknown whether such misfolding of proinsulin peptide is associated with T2D in the absence of INS mutations. Also, it is not known whether proinsulin misfolding is present in the early triggering stages of T2D, including prediabetes and mild dysglycemia prior to more obvious islet failure including β-cell degranulation and dedifferentiation.

### SUMMARY OF THE DISCLOSURE

The present invention includes methods relating to determining a likelihood of developing type II diabetes by detecting abnormalities in the process of proinsulin misfolding in the pancreatic islets. Thus, one inventive subject matter includes a method for determining a likelihood of developing type II diabetes in a subject by detecting the presence of an aberrant proinsulin complex in a sample of the subject, which is correlated with an insulin tolerance of the subject. Typically, the sample comprises a tissue, a tissue culture, a cell, a cell extract, or a bodily fluid, a pancretic islet tissue, and/or a pancreatic beta cell.

In some embodiments, the aberrant proinsulin complex comprises a misfolded proinsulin peptide, a reactive thiol group, and/or at least a dimer of proinsulin peptides. Where the aberrant proinsulin complex comprises at least a dimer of proinsulin peptides, it is contemplated that the dimer of the proinsulin peptides is formed by a Cys(B19)-Cys(B19) disulfide bond between two proinsulin peptides. It is contemplated that such aberrant proinsulin complex can be detected by contacting the sample with an antibody that is specific to a proinsulin peptide. In some embodiments, the antibody is CCI-17.

Additionally, the method may further comprise a step of determining a high likelihood of developing type II diabetes when the amount of the aberrant proinsulin complex exceeds a predetermined threshold. In some embodiments, the predetermined threshold is at least 10% or at least 30% of an amount of mature insulin protein in the sample. Additionally and/or alternatively, the predetermined threshold can be at least 10% or at least 30% of total proinsulin peptides in the sample. Additionally and/or alternatively, the predetermined threshold is at least 10% more or at least 30% more of the aberrant proinsulin complex compared to an amount of the aberrant proinsulin complex detected in a healthy subject.

In some embodiments, described herein are methods of determining a likelihood of developing type II diabetes in a subject by detecting an abnormality of an ER oxidoreductase in a sample of the subject, which is associated with a presence of an aberrant proinsulin complex. Typically, the sample comprises a tissue, a tissue culture, a cell, a cell extract, or a bodily fluid, a pancretic islet tissue, and/or a pancreatic beta cell. In some embodiments, the ER oxidoreductase is protein disulfide isomerase A1. Additionally and/or alternatively, the abnormality comprises a mutation, a reduced activity, a reduced expression, or an intracellular mislocalization.

In some embodiments, the aberrant proinsulin complex comprises a misfolded proinsulin peptide, a reactive thiol group, and/or at least a dimer of proinsulin peptides. Where the aberrant proinsulin complex comprises at least a dimer of proinsulin peptides, it is contemplated that the dimer of the proinsulin peptides is formed by a Cys(B19)-Cys(B19) disulfide bond between two proinsulin peptides.

Additionally, the method may further comprise a step of determining a high likelihood of developing type II diabetes when the abnormality of the ER oxidoreductase exceeds a predetermined threshold. In such embodiments, the predetermined threshold can be at least 20% of reduced expression of the ER oxidoreductase, and/or at least 20% of reduced activity of the ER oxidoreductase.

In some embodiments, described herein are methods of preventing beta cell dysfunction in a subject in need thereof by suppressing formation of an aberrant proinsulin complex by facilitating an activity of an ER oxidoreductase. Typically, the aberrant proinsulin complex comprises at least a dimer of proinsulin peptides. In some embodiments, the dimer of the proinsulin peptides is formed by a Cys(B19)-Cys(B19) disulfide bond between two proinsulin peptides. In some embodiments, the ER oxidoreductase is protein disulfide isomerase A1.

It is contemplated that the activity of the ER oxidoreductase can be facilitated by overexpressing the ER oxidoreductase in a pancreatic cell of the subject, and/or activating a signaling pathway associated with an expression of the ER oxidoreductase. In some embodiments, facilitation of the activity of the ER oxidoreductase can be combined with an anti-diabetic therapy, which may include metformin, acarbose, or combinations thereof.

### INCORPORATION BY REFERENCE

All publications, patents, and patent applications mentioned in this specification and exhibits are herein incorporated by reference to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawings will be provided by the Office upon request and payment of the necessary fee.

The novel features of the disclosed methods are set forth with particularity in the appended claims. A better understanding of the features and advantages of the disclosed methods will be obtained by reference to the following detailed description that sets forth illustrative embodiments, in which the principles of the invention are utilized, and the accompanying drawings of which:

FIGS. 1A-F show data detecting improperly folder proinsulins.

FIGS. 2A-D depict data regarding the formation of proinsulin disulfide-linked complexes.

FIGS. 3A-D show data of misfolding of proinsulin in human (and rodent) pancreatic islets.

FIGS. 4A-E show data of improper proinsulin folding from pharmacological or physiological alteration of the β-cell ER folding environment.

FIGS. 5A-C show data indicating that accumulation of improperly folded proinsulin and detection of ER stress response are early events in the development of diabetes in leptin receptor-deficient mice.

FIGS. 6A-C show data indicating that proinsulin Cys residues that contribute to covalent complex formation; molecular mass markers are noted.

FIGS. 7A-B show data indicating that proinsulin intermolecular disulfide crosslinking is promoted by Cys(B19).

FIG. 8 shows a schematic diagram of Islet dysfunction during the natural history of diabetes in the LepRdb/db mouse, as a model.

FIG. 9A shows data implicating that secreted proinsulin does not exhibit available free thiols.

FIG. 9B shows data implicating that presence of free thiols in a subpopulation of proinsulin molecules from mouse islet beta cells.

FIG. 9C shows data implicating that inhibition of PERK promotes formation of proinsulin disulfide-linked complexes in a rat beta cell line.

FIG. 10 shows presence of proinsulin disulfide-linked complexes in human islets.

FIG. 11A shows data indicating that inhibition of PERK promotes formation of proinsulin disulfide-linked complexes in human islets.

FIG. 11B shows data indicating loss of intact BiP promotes rapid formation of proinsulin disulfide-linked complexes.

FIG. 11C shows data of cleavage of BiP by SubAB. INS1E cells incubated without or with SubAB toxin were immunoblotted with anti-KDEL.

FIG. 12 shows data of intracellular proinsulin distribution in the LepRdb/db mouse.

FIG. 13 shows immunohistological data showing intracellular proinsulin distribution in the LepRdb/db mouse.

FIG. 14 shows data of presence of free thiols in recombinant proinsulin mutants.

FIG. 15A shows a schematic diagram of native intramolecular disulfide bonding of proinsulin.

FIG. 15B shows data indicating that some possible scenarios of intermolecular disulfide bonding of proinsulin mutant keep-B19/A20.

FIGS. 16A-D show data that conditional β cell-specific Pdia1 deleted mice were generated with Tamoxifen (Tam) induction.

FIGS. 17A-E show data indicating that Pdia1 is specifically and persistently deleted in murine β cells.

FIGS. 18A-I show data indicating that β cell-specific Pdia1 deleted male mice are glucose intolerant with defective insulin secretion when fed a 45% High Fat Diet (HFD).

FIGS. 19A-J show data indicating that Pdia1 deletion induces morphological abnormalities including decreased insulin granule numbers,

FIGS. 20A-F show data that Pdia1 deletion in HFD fed mice increases islet steady state proinsulin to insulin ratio with accumulation of high molecular weight (HMW) proinsulin complexes.

FIGS. 21A-F show data that Pdia1 deletion increases sensitivity to menadione oxidant: Increased ROS, nuclear condensation, and HMW proinsulin complexes were observed in menadione-treated KO islets.

FIG. 22 shows a schematic diagram illustrating the role of PDIA1 in proinsulin disulfide bond formation.

FIGS. 23A-B show that the RIP-Cre^{ERT} allele does not impact the β cell-specific Pdia1 deletion phenotype.

FIGS. 24A-B show that β cell area relative to pancreas area and cell number relative to islet area were not changed in β cell-specific Pdia1 deleted male mice after 34 wks of HFD.

FIG. 25 shows a table of primer sequences used for qRT-PCR.

FIGS. 26A-D show that Pdia1 deletion increases accumulation of HMW proinsulin complexes under regular diet.

FIG. 27 shows data indicating inhibition of ER to Golgi trafficking increases proinsulin disulfide linked HMW complex formation.

FIG. 28 shows that PDIA1 overexpression reduces proinsulin.

FIGS. 29A-B show that PDIA1 inhibitor KSC-34 recapitulates effects of Pdia1 deletion.

### DETAILED DESCRIPTION OF THE DISCLOSURE

### Definitions

As used in the specification and appended claims, unless specified to the contrary, the following terms have the meaning indicated below.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, “treatment of” or “treating,” ‘applying”, or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease or condition, or to a patient reporting one or more of the physiological symptoms of a disease, even though a diagnosis of this disease has not been made.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

Biosynthesis of insulin—critical to metabolic homeostasis—begins with folding of the proinsulin precursor, including formation of three evolutionarily conserved intramolecular disulfide bonds. Remarkably, normal pancreatic islets contain a subset of proinsulin molecules bearing at least one free cysteine thiol. As disclosed herein, in human (or rodent) islets with a perturbed endoplasmic reticulum folding environment, non-native proinsulin peptides enter intermolecular disulfide-linked complexes. In genetically obese mice with otherwise wild-type islets, disulfide-linked complexes of proinsulin are more abundant, and leptin receptor-deficient mice, the further increase of such complexes tracks with the onset of islet insulin deficiency and diabetes. As disclosed herein, proinsulin's Cys(B19) and Cys(A20) are necessary and sufficient for the formation of proinsulin disulfide-linked complexes; indeed, proinsulin Cys(B19)-Cys(B19) covalent homodimers resist reductive dissociation, highlighting a structural basis for aberrant proinsulin complex formation.

Viewed from a different perspective, as disclosed herein, misfolded proinsulin peptides, especially the aberrant proinsulin complexes having intermolecular disulfide-linked complexes can be a marker for early development of type II diabetes, thus can be used for diagnosis of early phase of type II diabetes. Thus, in some embodiments, described herein are methods of determining a likelihood of developing type II diabetes in a subject by detecting the presence of an aberrant proinsulin complex in a sample of the subject.

Any suitable samples, preferably biological samples, which include or may include proinsulin peptides and/or mature insulin proteins, are contemplated. Exemplary samples may include biological tissues (e.g., pancreatic tissues obtained from a biopsy), cells (e.g., dissociated cells obtained from biopsy tissue, etc), cultured cells (e.g., cell lines, etc.), cultured tissues, cell extract, and biological fluid (e.g., whole blood, serum, ascitic fluid, cerebrospinal fluid, urine, etc.). Additionally, samples may be fresh or frozen. Preferably, samples are prepared in a non-reduced condition (e.g., addition of dithiothreitol (DTT) to the sample preparation, etc.) such that any existing disulfide bonds on the proinsulin peptides or insulin proteins are preserved.

As used herein, the term “aberrant proinsulin complex” refers any peptide complex comprising proinsulin peptide(s) that fails to form any one of three intramolecular disulfide bonds (disulfide bonds between A and B polypeptide chains (A7-B7 and A20-B19) and one in the A chain (A6-A11)) to form a mature insulin protein. As a proinsulin peptide complex is likely to have a free thiol group (in one or more A6, A7, All, A20, B7, B19 residue) to form an intermolecular disulfide bonds between two proinsulin peptides (e.g., B19-B19 disulfide bonds), such aberrant proinsulin complexes include a dimer, a trimer, a tetramer, a pentamer, or any other types of multimer of proinsulin peptides formed by intermolecular disulfide bonds. Further, it is contemplated that at least some forms of such aberrant proinsulin complexes are resistant to forming mature insulin proteins. For example, B19-B19 disulfide bonds are essentially irreversibly formed in some aberrant proinsulin complexes to prevent the aberrant proinsulin complexes from reducing the B19-B19 disulfide bond and alternatively forming A20-B19 disulfide bond that is required for forming a mature and functional insulin protein.

Consequently, it is contemplated that detection of aberrant proinsulin peptides in the sample can be used to determine the likelihood of developing type II diabetes, or prognosis of early phase of type II diagnosis that cannot be easily determined by morphological or physical changes of samples (e.g., deformation of pancreatic β cells, significant changes in blood sugar level, loss of pancreatic β cells, etc.). As will be further described in the Examples, at least some aberrant proinsulin peptides can be specifically and distinctly labeled and detected in the samples using a binding molecule (e.g., an antibody) which binds to aberrant proinsulin complexes, yet does not bind to a mature insulin molecule. While exemplary and currently available antibodies that can be used for detecting aberrant proinsulin peptides are listed in the Examples, including mouse mAb CCI-17 that is directed an epitope in the region of the RQKRGIVEQ sequence of rat proinsulin (which spans the C-A cleavage junction) or mouse mAb directed against an epitope in the region of PLALEGSLQKRGIV sequence of human proinsulin (which spans the C-A cleavage junction), it is contemplated that any binding molecule that targets an epitope in a junction between chain B and chain C domains and/or a junction between chain C and chain A domains of the proinsulin peptide, that are cleaved out during maturation process of the insulin protein, can be used in the methods described herein.

Presence of aberrant proinsulin peptide complexes in the sample can be detected with any suitable method of detecting peptide or proteins. For example, aberrant proinsulin peptide complexes in the sample (e.g., tissue extracts, cell extracts, etc.) can be placed on a non-reducing SDS-PAGE and detected by western blotting using the antibody that can specifically and distinctly binds to the aberrant proinsulin peptides. Alternatively, aberrant proinsulin peptide complexes binding to the antibody in the sample can be isolated (e.g., using pull-down assay). In such examples, it is further contemplated that a relative amount of aberrant proinsulin peptide complexes to the mature insulin proteins can be measured by comparing the signal intensities in the western blot, or an absolute amount of aberrant proinsulin peptide complexes can be determined by measuring the concentration of the aberrant proinsulin peptide complexes pulled down with the antibody.

In another example, aberrant proinsulin peptide complexes in the sample (e.g., tissue slices, cultured cells, acutely dissociated cells, fixed tissues, etc.) can be detected using any visualization techniques (e.g., immunocytochemistry or immunohistochemistry using fluorescence or chemiluminescence labeling, followed by any suitable microscopy techniques, etc.). In some embodiments, a relative amount of an aberrant proinsulin peptide complexed to the mature insulin protein can be measured by comparing the signal intensities in the cells and/or tissues. Also, subcellular localization (e.g., in the ER, at the border of ER and Golgi apparatus, trans-Golgi network, lysosome, etc.) of the aberrant proinsulin peptide complexes can be determined and/or compared with the subcellular localization of the mature insulin proteins.

As disclosed herein, the amount and/or localization of such detected aberrant proinsulin peptide complexes can be used to determine the high likelihood of developing type II diabetes. In some embodiments, the likelihood of developing type II diabetes can be determined based on a predetermined threshold of the amount of aberrant proinsulin peptide complexes. For example, the likelihood of developing type II diabetes can be determined high when the amount of aberrant proinsulin peptide complexes exceeds 5%, 10%, 15%, 20%, 25%, 30%, 35%, 40%, 45%, or 50% of the mature insulin protein in the sample. In another example, the likelihood of developing type II diabetes can be determined high when the amount of aberrant proinsulin peptide complexes exceeds 5%, 10%, 15%, 20%, 25%, 30%, 35%, 40%, 45%, or 50% of the total proinsulin peptides present in the sample. Alternatively and/or additionally, the likelihood of developing type II diabetes can be determined by comparing the amount of aberrant proinsulin peptide complexes in the sample with those in the healthy subject (e.g., no sign or history of type II diabetes, no other health record related to the blood sugar level, etc.). In such example, the likelihood of developing type II diabetes can be determined high when the amount of aberrant proinsulin peptide complexes in the sample is at least 10%, 15%, 20%, 25%, 30%, 35%, 40%, 45%, or 50% more of the aberrant proinsulin complex compared to an amount of the aberrant proinsulin complex detected in a healthy subject.

Alternatively and/or additionally, as disclosed herein, abnormalities of an enzyme critical to the proper folding of proinsulin peptide can be a signature for early sign of type II diabetes development or a marker for determining a likelihood of developing type II diabetes in a subject. As further shown in detail in the examples, ER oxidoreductase, preferably, protein disulfide isomerase A1 (PDIA1/P4HB), which is one of the most abundant ER oxidoreductase of over 17 members, interacts with proinsulin to influence disulfide maturation and further is required for optimal insulin production under metabolic stress in vivo. In addition, β cell-specific Pdia1 deletion in young high-fat diet fed mice or aged mice exacerbated glucose intolerance with inadequate insulinemia and increased the proinsulin/insulin ratio in both serum and islets compared to wildtype mice. Ultrastructural abnormalities in Pdia1-null β cells include diminished insulin granule content, ER vesiculation and distention, mitochondrial swelling and nuclear condensation. Furthermore, Pdia1 deletion increased accumulation of disulfide-linked high molecular weight proinsulin complexes (aberrant proinsulin peptide complexes) and islet vulnerability to oxidative stress.

As used herein, an abnormality of the ER oxidoreductase may refer any mutations (e.g., deletion, insertion, substitution, duplication, etc.) in the gene(s) encoding the ER oxidoreductase, any abnormal expression (overexpression, underexpression, loss of expression, etc.) of the ER oxidoreductase in RNA transcription or protein translations, any abnormalities in the post-translational modifications that may affect the activity (e.g., phosphorylation, glycosylation, protein-protein interactions, etc.), any abnormalities of activities (e.g., loss or reduced catalytic activity of the individual or groups of ER oxidoreductase, subcellular mislocalization, abnormal clustering or aggregation, abnormal degradation, interference in the signaling cascade upstream or downstream of the ER oxidoreductase, etc.).

Any suitable methods to determine the abnormalities of the ER oxidoreductase are contemplated. For example, mutations in the ER oxidoreductase can be detected by sequencing of DNA or RNA encoding the ER oxidoreductase. In another example, an abnormal expression of ER oxidoreductase in the RNA level can be detected by quantitative RT-PCR. In still another example, an enzyme activity of the ER oxidoreductase can be measured using spectrophotometric assays.

As disclosed herein, such detected abnormalities of ER oxidoreductase can be used to determine the high likelihood of developing type II diabetes. In some embodiments, the likelihood of developing type II diabetes can be determined based on a predetermined threshold of the abnormalities of ER oxidoreductase. For example, the likelihood of developing type II diabetes can be determined high when RNA and/or protein expression level of the ER oxidoreductase in the sample is reduced at least 10%, 15%, 20%, 25%, 30%, 35%, 40%, 45%, or 50% compared to RNA and/or protein expression level of the ER oxidoreductase detected in a healthy subject. In another example, the likelihood of developing type II diabetes can be determined high when the catalytic activity of the ER oxidoreductase is reduced at least 10%, 15%, 20%, 25%, 30%, 35%, 40%, 45%, or 50% compared to catalytic activity of the ER oxidoreductase detected in a sample obtained from a healthy subject.

Provided that the formation of aberrant proinsulin peptide complexes causes the pancreatic beta cell dysfunction, and the reduced catalytic activity of the ER oxidoreductase or reduced expression of ER oxidoreductase is associated with such formation of the aberrant proinsulin peptide complexes, it is further contemplated that progression or development of type II diabetes can be prevented or at least delayed by suppressing formation of an aberrant proinsulin complex, and/or facilitating an activity of an ER oxidoreductase, and/or suppressing formation of an aberrant proinsulin complex by facilitating an activity of an ER oxidoreductase.

In some embodiments, the activity of the ER oxidoreductase can be facilitated by overexpressing the ER oxidoreductase in a pancreatic cell of the subject. For example, an expression construct can be generated with a wild-type ER oxidoreductase construct in a viral vector (e.g., E1 or E2b deleted adeno viral vector), and can be injected into the subject, or directly to the subject's pancreatic tissues using tissue-specific or cell-specific expression promoters. Additionally and/or alternatively, the activity of the ER oxidoreductase can be facilitated by activating a signaling pathway associated with an expression of the ER oxidoreductase. For example, the activity of the ER oxidoreductase can be facilitated by stimulating or expressing Protein kinase RNA-like endoplasmic reticulum kinase (PERK).

Additionally, it is further contemplated that the effect of facilitating ER oxidoreductase activity can be augmented by combining anti-diabetic therapy. Any suitable anti-diabetic therapies with glucose lowering effect are contemplated, including metformin, acarbose, biguanides, sulfonylureas, meglitinide, thiazolidinedione (TZD), dipeptidyl peptidase 4 (DPP-4) inhibitors, sodium-glucose cotransporter (SGLT2) inhibitors, and α-glucosidase inhibitors. In some embodiments, such anti-diabetic therapies can be treated to or provided to the subject concurrently with any treatment facilitating ER oxidoreductase activity. Alternatively and/or additionally, the anti-diabetic therapies can be treated to or provided to the subject at least before or after 24 hours, 48 hours, 3 days, 7 days, 14 days, 28 days, 2 months, 3 months, or 6 months after treating the subject with any treatment facilitating ER oxidoreductase activity.

### EXAMPLES

### Example I: Proinsulin Misfolding is an Early Event in the Progression to Type 2 Diabetes

Chemicals and Reagents: AMS, DTT, N-ethylmaleimide, PERK inhibitor (GSK2656157), MG132, Cycloheximide, and all other chemicals were purchased from Sigma-Aldrich or ThermoFisher Scientific. SDS-PAGE 4-12% Bis-Tris or 4-20% Tris-Glycine NuPage gels were obtained from ThermoFisher.

Antibodies: Antibodies used in this example are as follows: 1) mouse mAb 20G11 directed against an epitope in the region of EAEDLQVGQVELGG of human C peptide was obtained at the Scripps antibody production core facility. This mAb does not significantly cross-react with rodent proinsulin; 2) Mouse mAb GS-9A8 directed against an epitope in the region of YTPKTRREAEDL of human proinsulin (which spans the B-C cleavage junction and cross-reacts with rodent proinsulin in formaldehyde-fixed tissue) was obtained from the DSHB at the University of Iowa; 3) Mouse mAb CCI-17 directed an epitope in the region of the RQKRGIVEQ sequence of rat proinsulin (which spans the C-A cleavage junction) was obtained from ALPCO. This mAb does not significantly cross-react with human proinsulin; 4) Mouse mAb directed against an epitope in the region of PLALEGSLQKRGIV sequence of human proinsulin (which spans the C-A cleavage junction) was obtained from Abmart. This mAb shows partial cross-reactivity with rodent proinsulin; 5) guinea pig anti-insulin was used both for immunofluorescence (DAKO) and Western blotting (Covance). Guinea pig polyclonal anti-insulin cross reacts with proinsulin and conversion intermediates, but it preferentially reacts with mature insulin, of multiple species; 6) mouse mAb anti-KDEL was from Enzo Life Sciences; 7) Rabbit polyclonal anti-calnexin was as previously described in Kim, P. S., Kwon, O.-Y. & Aryan, P. J. Cell Biol. 133, 517-527 (1996); 8) mouse mAb anti-GM130 was obtained from BD Biosciences; 9) rabbit polyclonal anti-cyclophilin B was obtained from ThermoFisher; 10) rabbit mAb anti-p58ipk was obtained from Cell Signaling Technologies.

Mouse models: The mutant LepRdb allele was carried in the C57BLKS/J background; heterozygotes were crossbred to generate LepRdb/db homozygotes. Wild-type and Akita mutant mice were maintained in the C57BL/6J background. All of the preceding breeder mice were obtained from JAX. Ins1−/−, Ins2+/− mice generated in a C57BL/6 background were intercrossed as previously described in Duvillie, B. et al. Proc Natl Acad Sci USA 94, 5137-5140 (1997); Duvillie, B. et al. Endocrinology 143, 1530-1537 (2002). In this example, the analysis is restricted to males because Akita diabetic males develop progressive hyperglycemic to which the females are resistant. LepRNkx2.1 KO mice were identical to those previously described in Ring, L. E. & Zeltser, L. M. J Clin Invest 120, 2931-2941 (2010).

Mouse pancreatic islet isolation: Mice were euthanized by CO2 narcosis as per an approved institutional animal protocol. The pancreas was rapidly excised, minced in ice-cold PBS, and digested in 4 mL of Collagenase P (Roche) 1.5 mg/mL in Hank's Balanced Salt Solution containing calcium and magnesium, in a shaking water bath for 30 min at 37° C. The digestion was terminated in 40 mL ice-cold PBS (—Ca/—Mg) and the digested tissue washed twice in this buffer. The sedimented tissue digest was then overlaid with 3 mL Histopaque-1077 and further overlayed with 6 mL ice cold PBS (—Ca/—Mg) before centrifugation (3000 rpm for 20 min at 10° C. with no brake) and transfer of the midlayer of the Histopaque gradient to a 15 ml tube for two further washes in ice-cold PBS. Finally, the sedimented tissue was transferred to a petri dish in ice-cold RPMI-1640 buffer and islets hand-picked to purity. In experiments involving drug or compound treatment, the islets were finally recovered overnight in RPMI-1640 supplemented with 10% fetal bovine serum (FBS), 10 mM HEPES pH 7.35, 1 mM sodium pyruvate and 0.05 mM beta-mercaptoethanol, in a humidified 5% CO2 incubator at 37° C. For some experiments, islets were snap frozen in liquid nitrogen and stored frozen prior to analysis.

Human pancreatic islets: Human islets were obtained either from Prodo Labs, or from the NIDDK-funded Integrated Islet Distribution Program (IIDP; NIH UC4-DK098085) and maintained in a humidified 5% CO2 incubator at 37° C. Human islets were cultured ex vivo for up to 96 h in Prodo PIM(R) islet19 specific tissue culture medium supplemented with 10% FBS and Prodo PIM(G) glutamine/glutathione supplement, plus penicillin/streptomycin.

Cell and islet culture: Rat pancreatic beta cell lines INS1E and INS-832/13 were cultured in RPMI-1640 medium supplemented with 10% FBS, 10 mM HEPES pH 7.35, 1 mM sodium pyruvate, penicillin/streptomycin and 0.05 mM beta-mercaptoethanol. HEK293T human cells lines were cultured in DMEM supplemented with 10% FBS and penicillin/streptomycin.

AMS-mediated alkylation of proinsulin: INS1E cell or mouse or human islet lysate, each diluted in reaction buffer (50 mM Tris pH 7.4, 1% SDS final concentrations) were heated to 95° C. for 5 min, cooled to room temperature and then incubated further in the same buffer containing 4-Acetamido-4′-Maleimidylstilbene-2,2′-Disulfonic Acid, Disodium Salt (AMS, ThermoFisher) for 1 h at 37° C. In one experiment, 2 mM DTT was added during the initial boiling step prior to alkylation. No differences in alkylation were observed at AMS doses ranging from 6 mM to 20 mM. Non-alkylated controls underwent all the same incubations, in the same buffers, in parallel. The INS1E cells bathing media were also similarly tested for AMS-reactive proinsulin species. AMS-treated and untreated controls were analyzed by nonreducing or reducing SDS-PAGE and immunoblotting as described below.

Proinsulin mutagenesis, and plasmids: The generation of myc-tagged keep-B7/A7, keep-B19/A20, and keep-A6/A11 were described previously in Haataj a, L. et al. Diabetes 65, 1050-1060 (2016). These plasmids were used as templates for further mutagenesis to create 6 single-cysteine myc-tagged proinsulin mutants using the QuikChange site-directed mutagenesis kit (Agilent). All resulting plasmids encoding corresponding proinsulin mutations were confirmed by direct DNA sequencing. The expression plasmid encoding hPro-CpepMyc has been previously described in Haataj a, L. et al. J. Biol. Chem. 288,1896-1906 (2013).

Transfection of cells: 293T cells, INS1E cells, and INS-832/13 cells at 70-80% confluency were transiently transfected using Lipofectamine 2000 (ThermoFisher) as per the manufacturer's instructions. A medium change was performed 5 h post-transfection and the cells were lysed at 36 or 48 h.

Lysis of cells or islets for SDS-PAGE and electrotransfer: After removal of media, cells were washed once with ice-cold PBS and lysed in RIPA buffer (10 mM Tris pH 7.4, 150 mM NaCl, 0.1% SDS, 1% NP40, 2 mM EDTA) plus protease inhibitor/phosphatase inhibitor cocktail (Sigma-Aldrich) or directly in Laemmli gel sample buffer. Lysates in RIPA buffer were immediately spun at 10,000 rpm for 10 min at 4° C. and the supernatants analyzed further or stored at −80° C. Islets that had been quick frozen were placed on ice and RIPA buffer containing protease inhibitor cocktail and phosphatase inhibitor was added. Lysis was carried out by gently pipetting or by syringe through a 30G needle. Total protein concentration in the lysate was determined by BCA or Bramhall assay, and 5-10 μg of samples prepared in SDS sample were resolved by SDS-PAGE in 4-12% Bis-Tris NuPAGE gels (Invitrogen) at 200 V for 30 min. Nonreducing gels were incubated in a solution containing 20 mM dithiothreitol (DTT) for 10 min at room temperature prior to electrotransfer.

Two-dimensional (2-D) gel electrophoresis: A lane excised from the nonreducing slab gel loaded with lysate from INS1E cells treated with PERK inhibitor, was incubated for 15 min at room temperature in Tris-Glycine pH 6.8 plus 20 mM DTT. The lane was then laid horizontally on top of a 10% SDS-polyacrylamide resolving gel and sealed in place in stacking gel. A single-tooth comb was introduced at one end of the stacking gel to introduce a one-dimensional reduced sample of proinsulin containing cell lysate (which runs as proinsulin monomer). The gel was run at 200 V for 45 mins in Tris-Glycine buffer pH 8.8 with electrotransfer as described above.

Immunoblotting: Transfer membranes were rinsed once in TBST (15 mM Tris, 150 mM NaCl, 0.1% Tween-20), blocked using TST plus 5% BSA for 1 h at room temperature, and then washed four times (5 min each) in TBST. Primary antibody and secondary antibodies were diluted in TBST and each of 21 incubations was 1 h at room temperature followed by 4 washes in TBST. On occasion, primary antibodies were incubated overnight at 4° C. Secondary antibodies were all HRP-conjugates. Development of immunoblots used enhanced chemiluminescence (Immobilon, Millipore, or SuperSignal West Pico PLUS, Thermo Fisher Scientific) with images captured using a Fotodyne gel imager.

Immunohistochemistry/immunocytochemistry: Pancreatic paraffin sections were de-paraffinized with Citrisolv (Fisher Scientific) for 5 min at room temperature, re-hydrated in a series of 8 min ethanol incubations: 100%, 95%, 70%, and distilled water. Antigen retrieval was carried out using Retrieve-ALL 1 Universal pH 8 (BioLegend) and slides were heated in a microwave, cooled for 30 min at room temperature, and incubated once with PBS for 5 min. Blocking was performed in 150 μL blocking buffer (3% BSA prepared in TBS and 0.2% Triton X-100) per section for 2 h at room temperature. Blocking buffer was removed and 150 μL per section of primary antibody appropriately diluted in TBS plus 3% BSA and 0.2% Tween-20 was incubated overnight at 4° C. The primary antibody was removed and the slide washed twice with TBS/0.1% Tween-20. Then, 150 μL secondary antibody (1:500 dilution, prepared in antibody buffer) per section was incubated for 1 h at room temperature. Slides were washed thrice with TBS/0.1% Tween-20, mounted with a drop of prolong gold anti-fade reagent with DAPI (ThermoFisher) and a cover slip affixed. For immunocytochemistry, INS1E cells were grown in an 8-well Millicell® EZ SLIDE (Millipore-SIGMA). Cells were allowed to reach 60-80% confluency before addition of PERK inhibitor (PERKi) or vehicle. After 18 h of PERKi treatment, the medium was removed and the cells were fixed in 3.7% formaldehyde in PBS pH 7.4, for 20 min at room temperature, rinsed once with PBS, and permeabilized with 0.4% Triton X-100 in TBS for 20 min at room temperature. The cells were washed thrice with TBS and then incubated in blocking buffer as described above. Thereafter samples were incubated overnight at 4° C. with 100 μL of appropriately diluted primary antibody in 3% BSA/TBS/0.2% Tween. The cells received four 15-min washes in TBS, and then incubated with secondary antibody (1:500 dilution) for 1 h at room temperature. The cells were then washed thrice with TBS and finally mounted with a drop of Prolong Gold anti-fade reagent containing DAPI (ThermoFisher) and a cover slip affixed and the slide incubated in the dark for 24 h at room temperature. A similar immunocytochemistry protocol was followed for INS-832/13 cells after SubAB treatment (4 h).

### Proinsulin in the ER has Reactive Cysteine Thiols and is Predisposed to Aberrant Disulfide-Linked Complex Formation

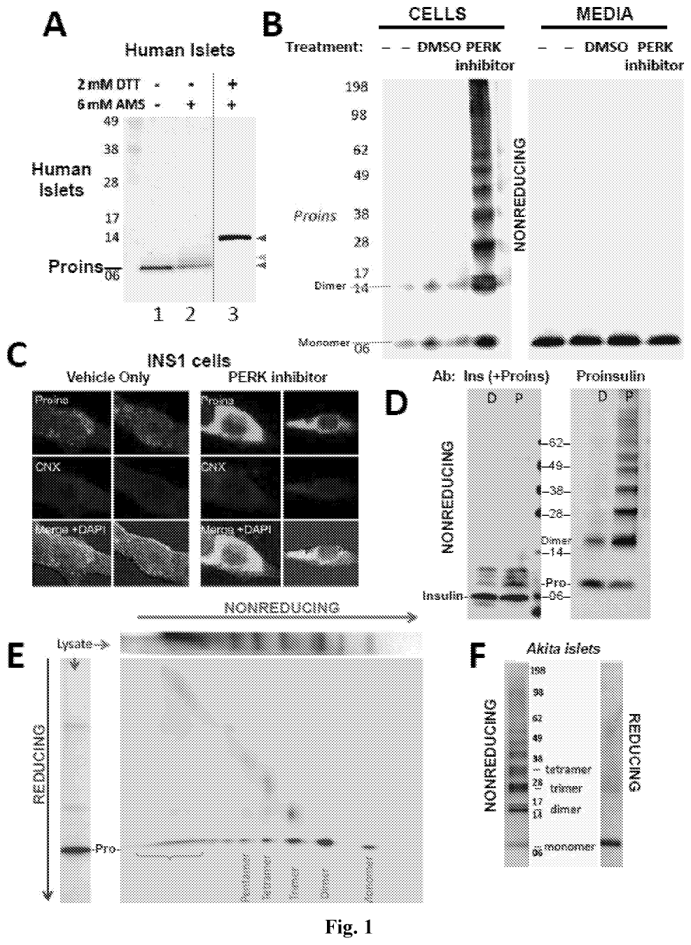

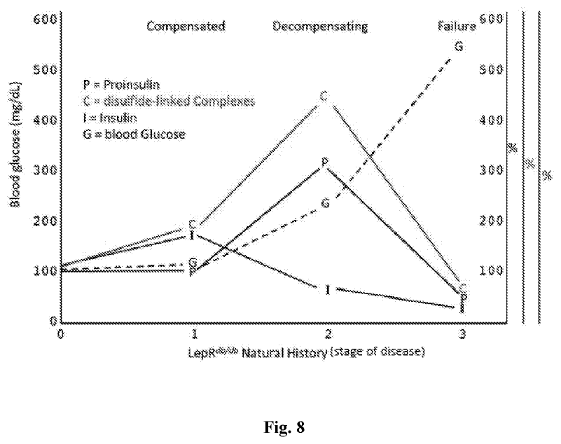

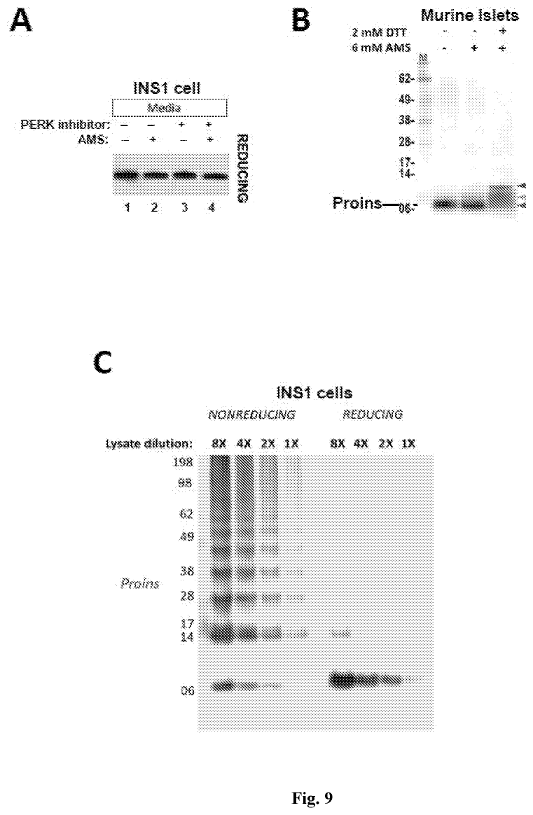

Both murine islets and the INS1 (rat) pancreatic β-cell line cells secrete successfully-folded proinsulin in addition to processed insulin. Native proinsulin folding requires formation of Cys(B7)-Cys(A7), Cys(B19)-Cys(A20) and Cys(A6)-Cys(A11) disulfide pairs. One way to detect improperly folded wild-type proinsulin in pancreatic (3-cells is to look for the possible presence of unpaired Cys residues. Alkylation of proinsulin Cys residues with 4-acetamido-4′-maleimidyl-stilbene-2,2′-disulfonate (AMS) adds 0.5 kD of molecular mass for each cysteine modified, shifting proinsulin from its normal molecular mass. FIG. 9A shows data implicating that secreted proinsulin does not exhibit available free thiols. Media from INS1E cells incubated overnight without or with PERK inhibitor (“+”) were collected, divided in half, and either not alkylated or alkylated with 10 mM AMS. The reactions were quenched with 200 mM DTT and resolved by SDS-PAGE in the presence of 200 mM DTT, electrotransferred to nitrocellulose, and analyzed by immunoblotting for rodent proinsulin with mAb CCI-17. No alkylated proinsulin was detected in the secretion, indicating that these molecules are properly folded. Remarkably, however, alkylation of intracellular proinsulin with AMS in human islets caused a decrease in unmodified proinsulin accompanied by the appearance of proinsulin alkylated on at least one cysteine thiol (FIG. 1A). In FIG. 1A, non-diabetic human pancreatic islets were lysed in RIPA buffer and divided into three equal parts, one of which was (partially) prereduced by boiling in the presence of 2 mM DTT (lane 3). This and a non-pre-reduced sample (lane 2) underwent alkylation with 6 mM AMS. A third sample was neither pre-reduced nor alkylated (lane 1). All samples were incubated for 1 h at 37° C. and finally resolved by SDS-PAGE under reducing conditions (200 mM DTT), electrotransfer to nitrocellulose, and immunoblotting for human proinsulin with mAb 20G11. The red arrowhead: proinsulin species with >1 alkylated Cys (upper band); beige arrowhead: at least 1 alkylated Cys (middle band); green arrowhead: no free Cys (bottom band). Alkylation of intracellular proinsulin was also observed in rodent islets as shown in FIG. 9B. FIG. 9B shows data implicating that presence of free thiols in a subpopulation of proinsulin molecules from mouse islet beta cells. Islets were lysed in RIPA buffer and divided into three equal parts, one of which was (partially) pre-reduced by boiling in the presence of 2 mM DTT (third lane). This and a non-pre-reduced sample (middle lane) underwent alkylation with 6 mM AMS. The sample in the first lane was neither pre-reduced nor alkylated. All samples were incubated for 1 h at 37° C. and finally resolved by SDS-PAGE under reducing conditions (200 mM DTT), electrotransfer to nitrocellulose, and immunoblotting for rodent proinsulin (“Proins”) with mAb CCI-17. The red arrowhead: proinsulin species with >1 alkylated Cys (upper band); beige arrowhead: at least 1 alkylated Cys (middle band); green arrowhead: no free Cys (bottom band). The presence of a free thiol in a significant subpopulation of proinsulin molecules can lead to inappropriate intermolecular disulfide attack on neighboring proinsulin molecules (Cunningham et al., 2017; Liu et al., 2010a; Liu et al., 2007; Wang et al., 1999).

Antibodies have been described that recognize misfolded proinsulin molecules bearing intermolecular disulfide bonds (e.g., Lee et al., 2016; Wang et al., 2011). Indeed, WT proinsulin is predisposed to misfolding. Proinsulin misfolding has not been demonstrated to be exacerbated in β-cells deficient for Ire1 or ATF6, but it has been repeatedly found to be exacerbated in β-cells with dysfunctional PERK (caused either by gene knockout, dominant-negative mutant, or specific chemical inhibitor)—leading to what has been described as the ‘proinsulin-impacted-ER’ phenotype (Gupta et al., 2010; Harding et al., 2012; Scheuner et al., 2005). The inventors performed immunoblotting of nonreducing SDS-PAGE samples with a monoclonal antibody that recognizes rodent proinsulin but not insulin (mAb CCI-17), with the intent to identify intermolecular disulfide-linked proinsulin complexes. As shown in FIG. 1B, Cell lysate (left) and overnight secretion (right) from untreated INS1E cells (-) or those treated with vehicle alone (DMSO) or PERK inhibitor (GSK2656157, 2 μM) were analyzed by nonreducing SDS-PAGE, electrotransfer to nitrocellulose, and immunoblotting for rodent proinsulin (mAb CCI-17). The positions of molecular mass markers are noted. Immunoblotting of either untreated β-cells or those treated with vehicle alone detected proinsulin monomers and disulfide-linked dimers (FIG. 1B left) the latter of which are, by definition, nonnative.

Moreover, whereas inhibition of PERK dramatically increased the intracellular accumulation of proinsulin and shifted its distribution to the classic impacted-ER phenotype (FIG. 1C), a remarkable ladder of higher molecular mass bands appeared upon Western blotting with mAb anti-proinsulin after nonreducing SDS-PAGE (FIG. 1B left). In FIG. 1C, INS1E cells were treated overnight with vehicle (DMSO) or PERK inhibitor before formaldehyde fixation, permeabilization, and indirect immunofluorescence with mAb anti-proinsulin (GS-9A8, green) and rabbit anti-calnexin (red), with appropriate secondary antibodies. Despite this increase of intracellular proinsulin, secretion of proinsulin to the medium was not increased; furthermore, by nonreducing SDS-PAGE, secreted proinsulin was recovered exclusively as the monomeric form (FIG. 1B right), demonstrating efficient quality control in the secretory pathway.

The inventors further observed that the intracellular accumulation of a ladder of proinsulin immunoreactive species upon nonreducing SDS-PAGE was not detected by conventional Western blotting with polyclonal anti-insulin, despite that this antibody identifies both proinsulin and proinsulin conversion intermediates (FIG. 1D left). However, dimers and higher order complexes were detected by Western blotting of the identical samples with mAb anti-proinsulin (FIG. 1D right), and all such higher bands, comprising the majority of intracellular proinsulin molecules, collapsed to monomers upon reduction of disulfide bonds (FIG. 9C). In FIG. 1D, INS1E cells were treated with DMSO (lane marked “D”) or PERK inhibitor (lane marked “P”). The cells were lysed and resolved in duplicate by nonreducing SDS-PAGE. The final gel was treated with 25 mM DTT for 10 minutes at 25° C., electrotransferred to nitrocellulose, and then immunoblotted with guinea pig anti-insulin that cross-reacts with proinsulin (“Pro”) and conversion intermediates (left panel) or anti-proinsulin (CCI-17, right panel). The positions of molecular mass markers are noted.

To confirm that the ladder of Western blotted bands detected by nonreducing SDS-PAGE specifically reflects disulfide-linked complexes of proinsulin, a similarly-generated sample was analyzed on a second-dimensional reducing SDS-PAGE, which demonstrated that nearly all bands in the nonreduced ladder contained proinsulin, with molecular masses expected of disulfide-linked complexes as simple as homodimers, homotrimers, homotetramers, and homopentamers, as well as higher order complexes (FIG. 1E). As shown in FIG. 1E, INS1E cells treated with PERK inhibitor as in panel B were lysed and resolved by a first dimensional nonreducing SDS-PAGE (shown horizontally, at top) and then in a second dimensional reducing SDS-PAGE (shown vertically, at left). The 2D gel was electrotransferred to nitrocellulose and immunoblotted for rodent proinsulin (mAb CCI-17). The bracket indicates high molecular weight proinsulin-containing complexes. Quantitatively these complexes comprised 87% of all recovered proinsulin. This is very similar to what has been reported for the islets of Akita mice that express misfolded proinsulin from one mutant allele that entraps wildtype proinsulin expressed from three wild-type alleles. Indeed, Western blotting of nonreduced lysates of Akita mouse islets with anti-proinsulin demonstrated that the majority of proinsulin was recovered in aberrant disulfide-linked proinsulin complexes (FIG. 1F), which were converted to monomeric proinsulin upon SDS-PAGE under reducing conditions.

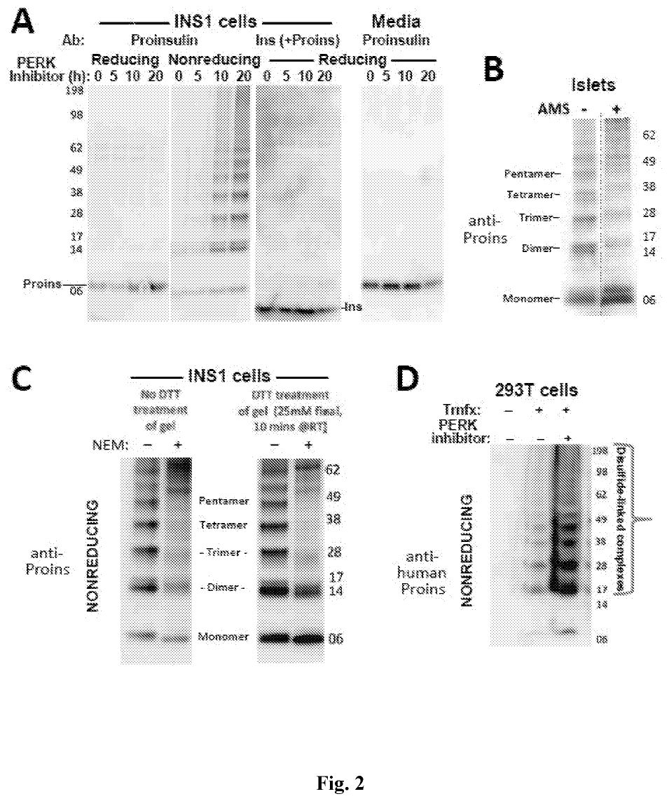

To confirm that even in the absence of INS gene mutations, formation of disulfide-linked proinsulin complexes constitutes improper proinsulin folding, INS1 pancreatic beta cells were exposed to PERK inhibitor for times up to 20 h. In detail, as shown in FIG. 2A, INS1E cells were incubated for 20 h in culture medium; the last 0, 5, 10, or 20 h of this incubation included PERK inhibitor as indicated. At the end of the 20 h, the media were collected and the cells were lysed; samples were resolved by nonreducing or reducing SDS-PAGE and electrotransferred to nitrocellulose. Panels 1, 2, and 4 were immunoblotted with mAb anti-proinsulin (CCI-17); panel 3 was immunoblotted with guinea pig anti-insulin. The positions of molecular mass markers are noted. With increasing time of exposure, the majority of intracellular proinsulin accumulated in disulfide-linked complexes in the β-cells (FIG. 2A second panel), and this increase was ultimately accompanied by decreased insulin content (third panel) as well as decreased proinsulin secretion (final panel). These observations are consistent with ER quality control limiting the ER export of misfolded proinsulin resulting in diminished delivery to post-Golgi sites (including the extracellular medium, also noted in FIG. 1B). Then, as shown in FIG. 2B, murine pancreatic islets treated overnight with PERK inhibitor were lysed in SDS gel sample buffer under nonreducing conditions, and divided into two portions. One portion of lysate was incubated with 6 mM AMS for 1 h, and then both portions were resolved by nonreducing SDSPAGE, electrotransferred to nitrocellulose, and immunoblotted with mAb anti-proinsulin (CCI-17). The availability of free thiols results in proinsulin bands shifting in the AMS-treated lysate (right) compared to the untreated one (left). The positions of molecular mass markers are noted. Significantly, treatment of intracellular proinsulin dimers and larger complexes with AMS, followed by nonreducing SDS-PAGE, revealed that each of the oligomeric species in the ladder of bands shifted up after alkylation, indicating that each of these forms still bears at least one unpaired cysteine thiol (FIG. 2B). These data provide a rationale for the propagation of disulfide-linked dimers into trimers, trimers into tetramers, etc.

The inventors further found that pretreatment of cells with NEM before (and during) cell lysis results in the detection of dramatically less of the ladder of disulfide-linked proinsulin oligomers (FIG. 2C)—and additionally, NEM pretreatment favors an increase in the detection of higher molecular weight proinsulin-containing complexes. As shown in FIG. 2C, INS1E cells treated with PERK inhibitor overnight were washed with ice cold PBS either lacking or containing 20 mM NEM and lysed in the absence (lane 1) or presence of 2 mM NEM (lane 2). These samples were run on two halves of the same nonreducing SDS-PAGE; the right half-gel was incubated for 10 min with 25 mM DTT, while the left half-gel remained untreated. Both halves were then electrotransferred and immunoblotted with mAb anti-proinsulin. DTT treatment of the gel increased the signal strength of proinsulin monomers (less so for dimers). Conversely, alkylation greatly decreased the signal strength of disulfide-linked proinsulin oligomers but yielded a similar ratio of distinct proinsulin oligomeric species to that seen without alkylation. Molecular mass markers are indicated. Nevertheless, the relative abundance of the distinct disulfide-linked oligomeric species of proinsulin was essentially unchanged in cells that were either alkylated in situ with 20 mM Nethyl maleimide prior to cell lysis, or not (FIG. 2C). These data render it unlikely that the detection of aberrant disulfide-linked complexes of nonmutant proinsulin is an artifact of cell lysis conditions.

Aberrant Disulfide-Linked Complex Formation of Proinsulin in Human Islets

To explore whether human proinsulin (FIG. 1A) is similarly predisposed to the aberrant disulfide-linked complex formation that was detected in rodent β-cells, recombinant human proinsulin was first expressed in heterologous cells, followed by nonreducing SDS-PAGE and immunoblotting with a monoclonal antibody that reacts exclusively with the amino-terminal region of human C-peptide. In detail, as shown in FIG. 2D, 293T cells were either mock-transfected or transfected (“Trnfx”) to express recombinant human proinsulin. Cells were treated overnight with vehicle (-) or PERK inhibitor before lysis, nonreducing SDS-PAGE, electrotransfer to nitrocellulose, and immunoblotting with mAb anti-human proinsulin (20G11). The positions of molecular mass markers are noted. Disulfide-linked proinsulin dimers along with a ladder of higher-order complexes already represented the majority of recombinant proinsulin species, and these forms accumulated to a dramatically higher level in cells treated overnight with PERK inhibitor (FIG. 2D).

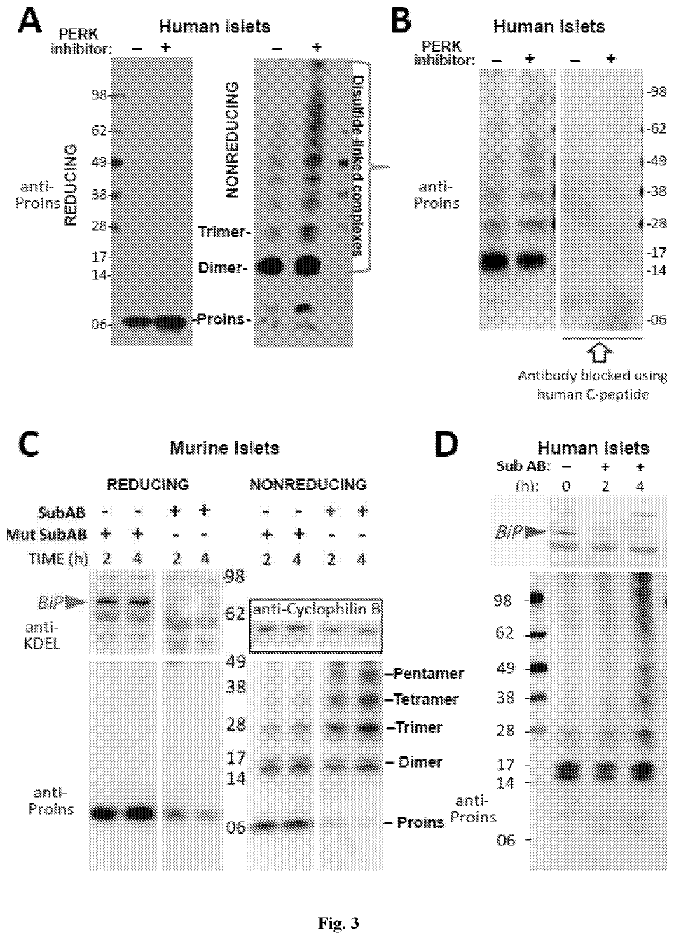

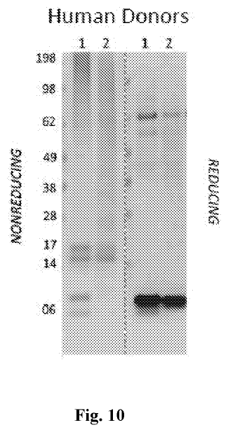

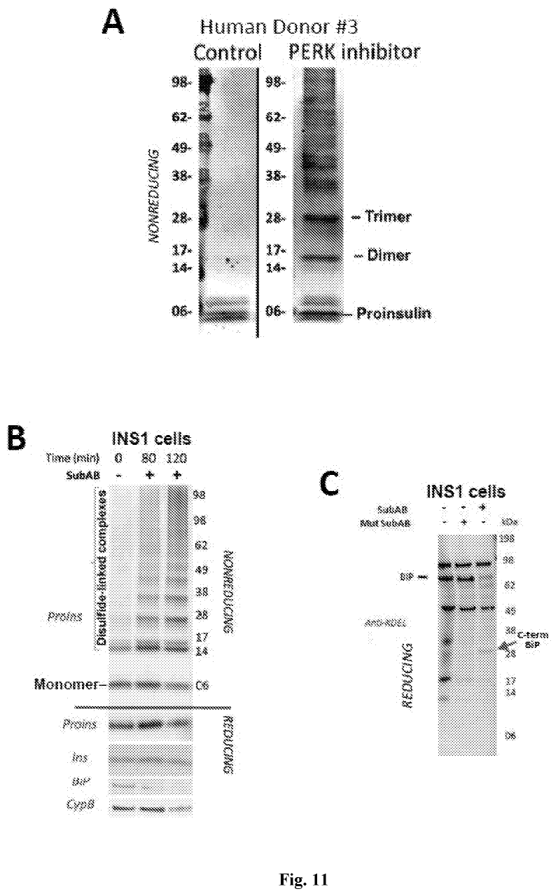

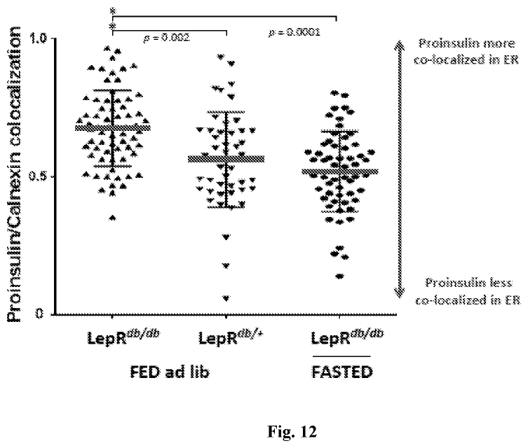

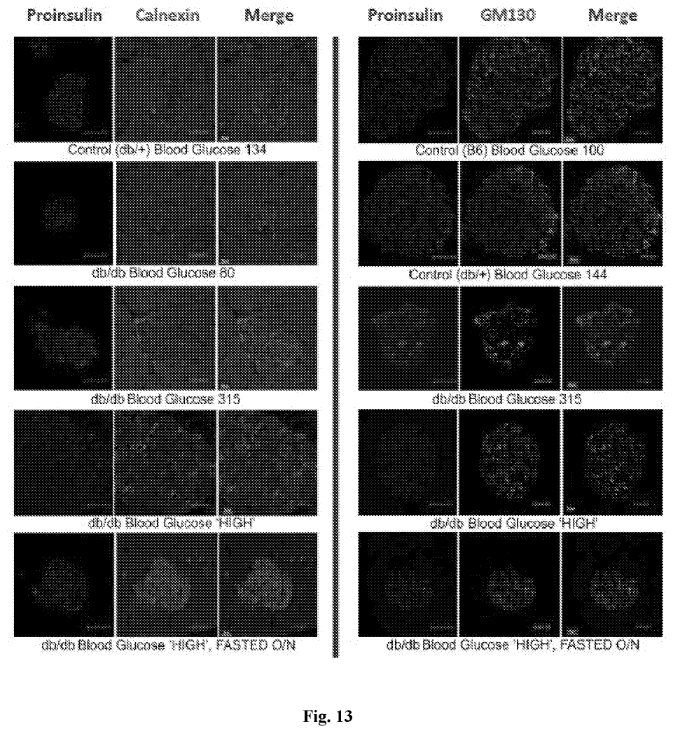

The inventors further looked for the presence of such disulfide-linked dimers and higher order complexes formed by endogenous proinsulin in preparations of human islets from unrelated donors without a history of T2D. As shown in FIG. 3A, Islets from humans not known to be diabetic were treated overnight with vehicle or PERK inhibitor before lysis, reducing or nonreducing SDS-PAGE, electrotransfer to nitrocellulose, and immunoblotting for human proinsulin (mAb 20G11). In several preparations, disulfide-linked dimers were prominent, along with a lesser abundance of higher-order complexes (FIG. 3A right panel). In human islets obtained from other individual donors, disulfide-linked dimers were less prominent (FIGS. 10 and 11). Loss of intensity of the intact BiP band (˜78 kD) coincided with the appearance of a ˜28 kD BiP fragment (“C-term BiP”) as has been noted previously (Paton et al., 2006). Overnight treatment of human islets with PERK inhibitor resulted in a further increase in higher order disulfide-linked complexes (FIG. 3A right panel, and FIG. 11) and an increase in total islet proinsulin as revealed by reducing SDS-PAGE (FIG. 3A left panel).

In detail, the inventors note that FIG. 10 shows presence of proinsulin disulfide-linked complexes in human islets. Lysates of human islets from two different donors were analyzed by nonreducing or reducing SDSPAGE and processed as in panel C using mAb anti-human proinsulin (20G11). FIG. 11A shows data indicating that inhibition of PERK promotes formation of proinsulin disulfide-linked complexes in human islets. Live islets from a human donor were incubated overnight in the absence of presence of PERK inhibitor (GSK2656157, 2 μM) and analyzed by nonreducing SDS-PAGE, electrotransfer to nitrocellulose, and immunoblotting with mAb antihuman proinsulin (20G11). FIG. 11B shows data indicating loss of intact BiP promotes rapid formation of proinsulin disulfide-linked complexes. INS1E cells were either control or incubated for the indicated short periods with SubAB toxin before cell lysis and immunoblotting for the antigens shown. Proins=proinsulin (below the red line show samples analyzed under reducing conditions in which all proinsulin migrates as a monomer). Ins=insulin; BiP=GRP78; CypB=Cyclophilin B (loading control). FIG. 11C shows data of cleavage of BiP by SubAB. INS1E cells incubated without or with SubAB toxin were immunoblotted with anti-KDEL.

These data indicate that aberrant disulfide-linked proinsulin dimers and higher complexes exist in human islets and their abundance responds to changes in environmental conditions within the ER. The immunodetection of both disulfide-linked dimers and higher-order complexes was blocked when pure human C-peptide competitor was included in the immunoblotting protocol (FIG. 3B), demonstrating specificity.

PERK inhibitor has a rapid onset of action on its target, but it appears that the impact of this inhibition to globally alter the proinsulin folding environment of the ER may take a half-day or more (FIG. 2A). More immediately, nascent proinsulin binds the hsp70 family member, BiP (Liu et al., 2005; Scheuner et al., 2005); thus, for a more direct perturbation, islets were exposed to the bacterial SubAB protease that is endocytosed and retrieved to the ER lumen where it cleaves the ER chaperone BiP within a matter of a few hours or less. In detail, as shown in FIG. 3B, mouse pancreatic islets were isolated and maintained in an overnight recovery medium including 11.1 mM glucose. The islets were then incubated with active SubAB (1 μg/mL final) or the same concentration of inactive mutant SubAA272B for 2 h or 4 h, as indicated. At each time point the islets were lysed and analyzed by reducing or nonreducing SDS-PAGE and immunoblotting with rabbit polyclonal anti-KDEL (recognizing multiple ER resident proteins including BiP, as indicated), mAb anti-proinsulin (CCI-17, lower panels), and anti-cyclophilin B (loading control, boxed). In mouse islets, BiP was ≥90% destroyed within 2 h after SubAB addition (FIG. 3C upper left), and immunoblotting revealed that this treatment shifted the majority of proinsulin into larger disulfide-linked complexes (FIG. 3C lower right). Similarly, in a rodent β-cell line, improperly folded proinsulin began to migrate increasingly in disulfide-linked complexes 80 min after SubAB addition when only a portion of intracellular BiP had been destroyed (FIG. 11). BiP cleavage was also confirmed by appearance of the ˜28kD C-terminal BiP cleavage fragment (FIG. 11).

In the islets of humans not known to have T2D, improperly folded disulfide-linked proinsulin dimers were already apparent prior to SubAB addition, and an increase in larger-sized covalent proinsulin complexes was apparent at 4 h of SubAB treatment (FIG. 3D lower panel). In FIG. 3D, islets from humans not known to be diabetic (obtained from the IIDP) were incubated with active SubAB (1.5 μg/mL final) for 0 h, 2 h or 4 h, as indicated. At each time point the islets were lysed and analyzed by nonreducing SDS-PAGE and immunoblotting with rabbit polyclonal anti-KDEL (upper panel) and mAb anti-proinsulin (20G11, lower panel). These data demonstrate an acute increase in improper folding of human proinsulin, without any INS gene mutation, under conditions of BiP deficiency within the β-cell ER luminal environment.

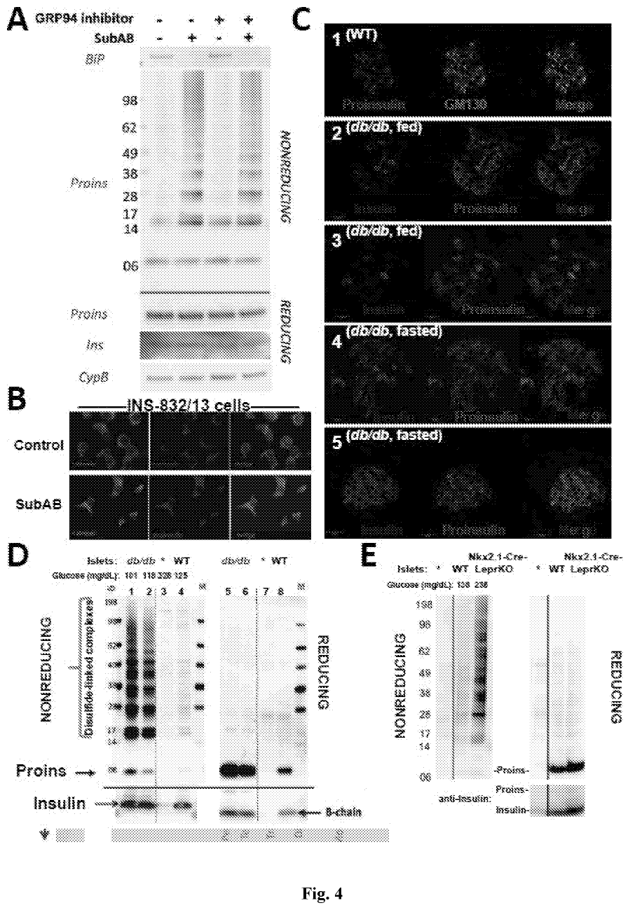

The hsp90 family member of the ER, GRP94, also impacts on proinsulin handling in β-cells, especially resulting in aberrant post-ER processing with markedly abnormal appearing secretory granules; however, treatment of β-cells with GRP94 inhibitor (PU-WS13, 20 μM) even for 24 h did not promote proinsulin disulfide-linked complex formation, and did not exacerbate the proinsulin disulfide-linked complex formation that was triggered by an acute (3 h) loss of BiP (FIG. 4A). In the experiments shown in FIG. 4A, INS1E cells were incubated for 24 h±20 μM GRP94 inhibitor (PU-WS13). During the last 3 h, SubAB was added where indicated. Cell lysates were analyzed by immunoblotting for BiP (top panel), and mAb anti-proinsulin (CCI-17) under nonreducing conditions (above red line) or with anti-proinsulin (Proins), anti-insulin (Ins), and anti-cyclophilin B (CypB, loading control) under reducing conditions (below red line).

INS832/13 incubated±active SubAB (1.5 μg/mL, 4h) were processed for immuno-fluorescence with rabbit anti-calnexin (green) or mAb anti-proinsulin (red). Immunofluorescence microscopy demonstrated that after SubAB treatment of β-cells, the intracellular distribution of proinsulin shifted from its usual predominant juxtanuclear localization in the Golgi region from which newly-made insulin granules emerge to a co-localization with the ER marker, calnexin (FIG. 4B). Altogether, these data strongly indicate that proinsulin disulfide-linked complexes are misfolded and are retained within the ER compartment.

### The Dynamics of Proinsulin Folding and Intracellular Distribution in a T2D Model

Sections of wild-type or LepRdb/db mouse pancreas (C57BL/6) were deparaffinized and prepared for indirect immunofluorescence. 1) Wild-type islets immunostained with mAb anti-proinsulin (CCI-17, in red), or the Golgi complex labeled with anti-GM130 (in green). 2-5) LepRdb/db mice with random blood glucose >500 mg/dL, as follows. 2 and 3) Mice fed ad lib; immunostained for insulin (blue) and mAb anti-proinsulin (CCI-17, red). 4 and 5) Mice fasted overnight, and immunostained as above. The third panel in each case is a merged image of the single-channel fluorescence. The inventors note that the predominant juxtanuclear Golgi-regional distribution of proinsulin is a feature of normal rodent islets (FIG. 4C first set of panels). Within the first 4-5 months of life, leptin receptor mutant LepRdb/db mice in a C57BL/6 background become severely diabetic and develop a paucity of islet β-cells immunostainable for mature insulin—indeed, the fraction of islet β-cells that exhibit little or no insulin immunostaining is known to increase by more than 5-fold compared to the islets of age-matched control mice. However, the inventors found that proinsulin-positive immunostaining amongst the cells in these diabetic islets was more widespread and the intracellular distribution of proinsulin within these islet β-cells was no longer as concentrated in the Golgi region (FIG. 4C second and third set of panels) with increased localization in the ER (FIG. 12). It has been established that limiting food intake of these animals can substantially increase the percentage of strongly insulin-immunopositive β-cells per islet (Ishida et al., 2017). Indeed, upon fasting overnight, the islets of diabetic LepRdb/db mice exhibited a more robust immunostaining of mature insulin (FIG. 4C fourth and fifth set of panels). Additionally, after fasting, there was a shift in intracellular proinsulin back towards a juxtanuclear distribution (FIG. 4C fourth and fifth set of panels). FIG. 12 shows data of intracellular proinsulin distribution in the LepRdb/db mouse. Sections of homozygous diabetic LepRdb/db or heterozygous LepRdb/+ (control) mouse pancreas from conditions of FIG. 4C (fed ad lib or fasted overnight) were immunostained with anti-proinsulin (CCI-17 as in FIG. 4C) and rabbit polyclonal anti-calnexin (ER marker) using two color immunofluorescence. Proinsulin-positive cells were examined for extent of proinsulin colocalization with the ER using by Manders' method using ImageJ software analyzed with “Coloc2” algorithm. Statistical analyses were performed with GraphPad Prism (version 7.00) using one-way ANOVA (Kruskal-Wallis) with Dunn's multiple comparison test to compare the sum of ranks. Data are individual pancreatic β-cells with mean plus standard deviation (SD) with p values shown.

The data of FIG. 4 suggests that in ad lib fed LepRdb/db diabetic mice, initial insulin depletion does not preclude ongoing proinsulin expression; moreover, a shift of proinsulin distribution towards an ER-like pattern (FIG. 12) may suggest a stressed state that may be preventable and to some extent reversible by limiting β-cell secretory stimulation (i.e., “β-cell rest”).

The inventors determined whether the ER-like distribution of intracellular proinsulin in LepRdb/db mice might be explained by (nonmutant) proinsulin folding exceeding the ER folding capacity of these β-cells, resulting in the accumulation of improperly folded species. With this in mind, proinsulin immunoblotting was performed after nonreducing and reducing SDS-PAGE in LepRdb/db islet lysates, with islet lysates from wild-type mice, or those bearing only one of 4 functional Ins alleles, as controls. As shown in FIG. 4D, isolated islets from young male LepRdb/db mice (lanes 1, 2, 5 and 6; random blood glucose values shown above) or wild-type C56BL/6 (“WT”, lanes 4, 8) or a high-fat fed Ins1+/−,Ins2−/− male (lanes 3 and 7, marked with asterisk) were lysed in RIPA buffer and analyzed by nonreducing or reducing SDS-PAGE and immunoblotting with mAb anti-proinsulin (CCI-17, above red line) or guinea pig anti-insulin (below red line). Under reducing conditions, >99% of proinsulin was monomeric (and insulin was recovered as the reduced insulin B-chain, FIG. 4D right). It was further examined proinsulin in the islet lysates of 5 additional prediabetic LepRdb/db mice—all were similar to the islets of two animals shown in FIG. 4D (4 and 6 weeks of age with random blood glucose levels of 101 and 118 mg/dL, respectively) containing a large steady state level of proinsulin (seen upon Western blotting of reducing SDS-PAGE) with >90% of molecules entangled in disulfide-linked dimers and higher-order complexes (seen by Western blotting upon nonreducing SDS-PAGE, FIG. 4D). Accumulation of misfolded proinsulin in the ER of LepRdb/db β-cells was always present before the onset of diabetes. These bands are indeed proinsulin, as they were absent from islets of mice depleted of three of four Ins alleles; FIG. 4D lane 3). Importantly, improperly folded proinsulin was also detectable in WT mouse islets, albeit at a much lower abundance (FIG. 4D, lane 4).

Because leptin receptors might have direct actions on β-cells, the inventors also examined islets from LepR-Nkx2.1 KO mice that are deficient for leptin receptor in the hypothalamus but not in the islets (Ring and Zeltser, 2010). As shown in FIG. 2E, isolated islets from WT and Nkx2.1-Cremediated LepR-KO mice (random blood glucose values shown above) were lysed in RIPA buffer and analyzed under nonreducing or reducing conditions as in panel D. Molecular mass markers are noted. Asterisk denotes lysate from Ins1+/-,Ins2-/- islets as in panel D. The positions of molecular mass markers are noted. Islet lysates from WT and Nkx2.1-Cre-mediated LepR KO mice were also immunoblotted with guinea pig anti-insulin (bottom right) that weakly cross reacts with proinsulin (Proins). Here too, pancreatic islets from these hyperphagic, obese, glucose intolerant animals demonstrated markedly increased abundance of disulfide-linked proinsulin complexes with >90% of proinsulin entangled in such complexes (FIG. 4E). These data indicate that an increase in improper proinsulin folding in leptin receptor-deficient animals is secondary to a prediabetic state and unrelated to leptin receptor function in β-cells. Crucially, these data also demonstrate that improper proinsulin folding is already detectable at a time that has been associated with ER stress response but before onset of chronic hyperglycemia associated with pancreatic insulin deficiency that has been attributed to a loss of functional β-cell mass regardless of whether this is due to de-differentiation or β-cell death.

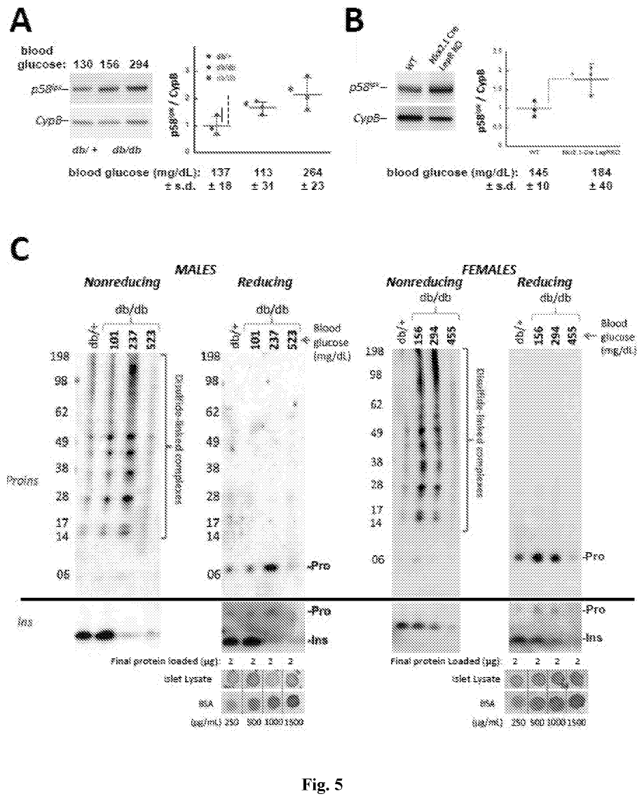

Next, it was further examined whether islet protein content of p58ipk (encoded by DNAJC3) is a reliable marker of islet ER stress response. In the experiment shown in FIG. 5A, islet lysates from LepRdb/+heterozygote (control) or LepRdb/db mice at different stages of diabetes progression (random blood glucose values shown above) were immunblotted for p58ipk and cyclophilin B (CypB, loading control); the graph at right shows the quantitation of the p58ipk/CypB ratio from three independent experiments (mean±s.d., each point a different animal; asterisk, p=0.05 by Mann Whitney U test and p<0.05 by t-test). In the experiment shown in FIG. 5B, islet lysates from WT and Nkx2.1-Cre-mediated LepR-KO mice were immunblotted for p58ipk and CypB as in panel A; the graph at right shows quantitation of the p58ipk/CypB ratio from three independent experiments (mean±s.d., each point a different animal; p=0.05 by Mann Whitney U test and p<0.05 by T test). In the experiment shown in FIG. 5C, islet lysates from LepRdb/+ heterozygote (control) or LepRdb/db mice at different stages of diabetes progression (random blood glucose values shown above; blood glucose in LepRdb/+male was 138 mg/dL and in female was 117 mg/dL) were analyzed by nonreducing or reducing SDS-PAGE and immunoblotting with mAb anti-proinsulin (CCI-17, above black line; molecular mass markers are noted) or guinea pig anti-insulin (below black line). Islet protein content from each mouse was measured relative to known BSA standards shown at bottom; 2 μg islet protein was analyzed for each sample. Left sets of gels are from males; right sets of gels are from females, as indicated. In LepRdb/db mice, intra-islet abundance of misfolded disulfide-linked proinsulin complexes reached maximum with random blood glucoses ranging from 150 -300 mg/dL but this was accompanied by a decline of intra-islet mature insulin levels. Overtly diabetic animals (random blood glucose >450) exhibited low insulin levels and ultimately exhibited low proinsulin levels as well. Thus, the inventors found that the levels of this ER co-chaperone of BiP began to increase in ad lib fed LepRdb/db mice even before the animals have reached a random blood glucose of 160 mg/dL (FIG. 5A). Islet p58ipk protein levels also tended to be elevated in LepR-Nkx2.1 KO mice (FIG. 5B).

In the islets of young, normoglycemic homozygous LepRdb/db male mice that are destined for diabetes, proinsulin disulfide-linked complexes (FIG. 5C upper left panel) were increased slightly above the level observed in heterozygous LepRdb/+ males (that do not progress to diabetes), and islet insulin levels were similarly increased (lower left panels). With a random blood glucose level in the mid 200′s (mg/dL), islet proinsulin was notably increased (FIG. 5C upper second panel) and comprised >90% of disulfide-linked proinsulin complexes (upper first panel) whereas simultaneously, islet insulin levels were dramatically decreased (lower left panels). With a random blood glucose level >500 mg/dL, islet proinsulin and insulin levels were both severely diminished (FIG. 5C left panels). In female LepRdb/db mice with a random blood glucose ranging from 156-294 mg/dL, insulin levels declined compared to the LepRdb/+control (FIG. 5C lower right panels). However, for females in this glycemic range, as in males, islet proinsulin was increased (FIG. 5 upper right panel) and once again, >90% was entangled in disulfide-linked proinsulin complexes (FIG. 5 upper third panel). Additionally, with a random blood glucose of 455 mg/dL, both proinsulin and insulin were severely diminished (FIG. 5C last two sets of panels) indicating islet decompensation.

FIG. 13 shows immunohistological data showing intracellular proinsulin distribution in the LepRdb/db mouse. Left side shows double immunofluorescence with mAb anti-proinsulin (red) and rabbit anti-calnexin (green) in islets of heterozygous LepRdb/+(control) and homozygous LepRdb/db mice with various levels of blood sugar indicative of worsening diabetes. The cytoplasmic proinsulin distribution tended to broaden towards the ER marker as a function of worsening glycemic control. The bottom panel involves fasting overnight as in FIG. 4C. Right side shows double immunofluorescence with mAb anti-proinsulin (red) and mouse anti-GM130 (green, using sequential blocking protocol) in islets of C57BL/6 (B6, control), heterozygous LepRdb/+(control), and homozygous LepRdb/db mice with various levels of blood sugar indicative of worsening diabetes. Under normoglycemic conditions, proinsulin tended to distribute more strongly in the Golgi region, with some apparent recovery of this distribution also seen in the islet β-cells of frankly diabetic animals after overnight fast (bottom panel). As indicated in FIG. 13, during this progression to diabetes, intracellular proinsulin distribution that initially displayed a prominent juxtanuclear pattern with good co-localization to the Golgi region (GM130 Golgi marker) tended to shift towards a staining pattern that was more spread in the cytoplasm, with increased co-localization with the ER marker, calnexin; although even in the fully diabetic state there appeared to be increased juxtanuclear (Golgi-like) proinsulin distribution after overnight fasting.

### Mechanism of Proinsulin Intermolecular Disulfide Complex Formation

Cys(A20) and Cys(B19) are two of the three most reactive thiols of proinsulin, and they initiate covalent association of B- and A-domains that are required for proinsulin export from the ER. Nevertheless, in the redox environment of the pancreatic β-cell ER, a human mutant proinsulin that retains only two cysteines, named ‘keep-B19/A20’ cannot undergo efficient intramolecular oxidation. Reactive free thiol availability (FIG. 1A) is a key to proinsulin participation in intermolecular disulfide-linked complex formation.

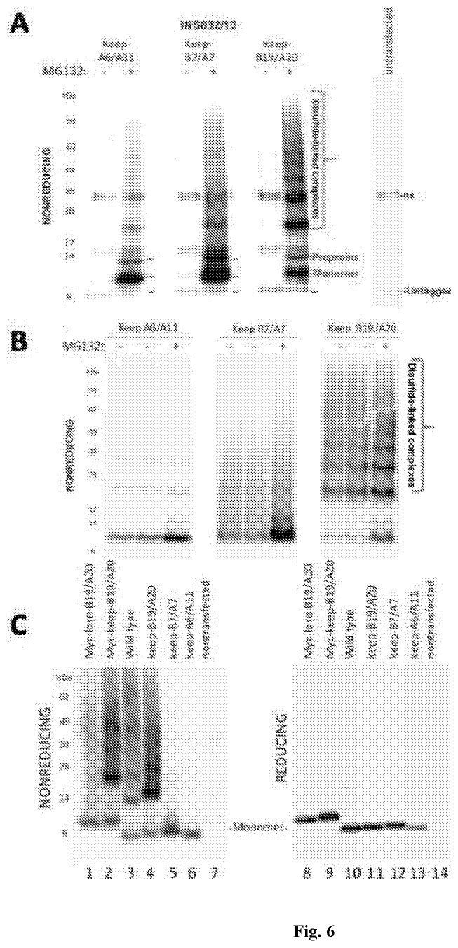

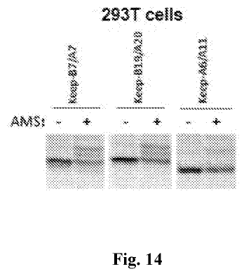

FIG. 14 shows data of presence of free thiols in recombinant proinsulin mutants. 293T cells transiently transfected to express recombinant myc-tagged proinsulin mutants known as keep-B7/A7, keep-B19/A20, or keep-A6/A11 were metabolically labeled with 35S-amino acids for 30 min, lysed in RIPA buffer, immunoprecipitated with anti-myc, alkylated with 40 mM AMS for 1 h at room temperature, and then boiled in SDS-gel sample buffer containing 200 mM DTT. The gels, analyzed by autoradiography, demonstrate that each of the 2-Cys proinsulin mutants has available free thiols that can react with the alkylating agent, causing upward band shift. Indeed, each of the myc-tagged human proinsulins keep-B7/A7 and keep-A6/A11, in addition to keep B19/A20, exhibited free thiol availability as determined by alkylation with AMS (FIG. 14). The inventors expressed each of these recombinant proinsulin mutants in the INS-832/13 β-cell line (which already expresses endogenous proinsulin). Because these two-cysteine mutant human proinsulins cannot be exported from the ER and are excellent ERAD substrates in pancreatic β-cells, these molecules were largely undetected by immunoblotting. However, in β-cells treated with proteasome inhibitor (MG132 for 7 h), myc-tagged proinsulin monomers were detected in all cases (FIG. 6A).

Remarkably, not only were these constructs capable of forming disulfide-linked proinsulin homodimers, but the keep-B19/A20 construct in particular recapitulated the situation in which the majority of molecules migrated as disulfide-linked proinsulin complexes (FIG. 6A). With or without MG132, this behavior was also observed upon expression in 293T cells that lack endogenous proinsulin (FIG. 6B), and these complexes appeared quite similar to those observed in human islets (FIG. 3A, B). In the experiment shown in FIG. 6A, INS832/13 were transfected to express myc-tagged recombinant human proinsulin “keep-A6/A11”, “keep-B7/A7”, or “keep-B19/A20” constructs. At 48 h post-transfection, cells were treated with vehicle alone (-) or MG132 (10 μg/mL) for 7 h before cell lysis and analysis by nonreducing SDS-PAGE and immunoblotting with mAb directed against a sequence (PLALEGSLQKRGIV) spanning the junction of the proinsulin C-peptide and insulin A-chain. A control of mock-transfected cells (far right lane) is shown for comparison; “ns”=nonspecific band. The positions of molecular mass markers are noted. In the experiment shown in FIG. 6B, 293T cells were transfected to express the same constructs and treated and analyzed as in panel A.

To determine if Cys(B19)/Cys(A20) are both necessary and sufficient for generating the full ladder of disulfide-linked proinsulin complexes, the three different human mutant proinsulins noted above that each retain only one set of potential disulfide partners were compared with a human proinsulin mutant named ‘lose-B19/A20’ in which Cys(B19) and Cys(A20) are mutated whereas positions B7, A7, All and A20 remain as cysteines. Once again, of the mutant human proinsulins that retain only one set of potential disulfide partners, only keep-B19/A20 could efficiently recreate the entire ladder of disulfide-linked complexes similar to that seen for wildtype proinsulin, irrespective of the presence or absence of an engineered myc epitope tag in the C-peptide (FIG. 6C left panel). However, lose-B19/A20 did not efficiently recreate the ladder of disulfide-linked complexes despite entrapment in the ER in a proinsulin construct bearing four Cys residues. These data indicate that the Cys(B19)/Cys(A20) pair is both necessary and sufficient for efficient propagation of disulfide-linked complexes of improperly-folded proinsulin. In the experiment shown in FIG. 6C, 293T cells were transfected to express the same constructs as in panel A, or myc-tagged “lose-B19/A20”, myc-tagged “keep-B19/A20”, and untagged wild-type human proinsulin. Cell lysates were resolved by SDS-PAGE under nonreducing and reducing conditions.

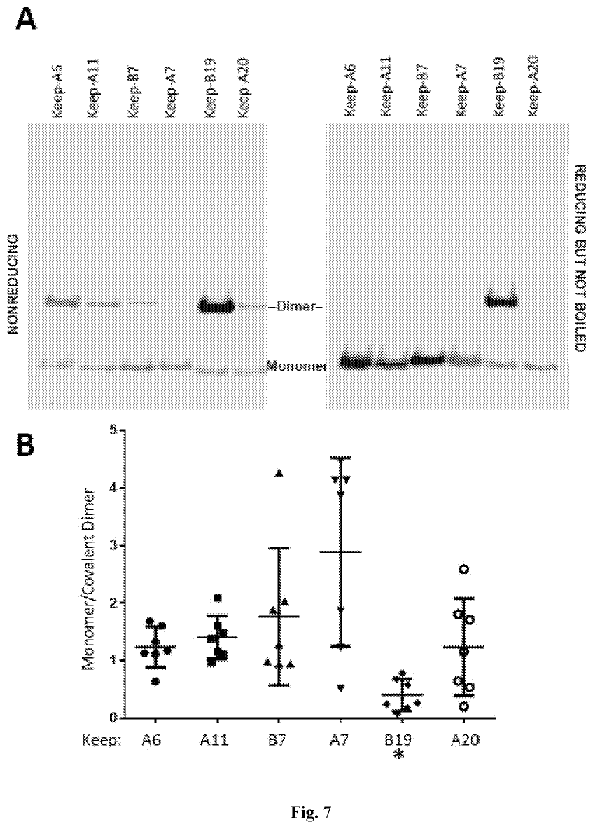

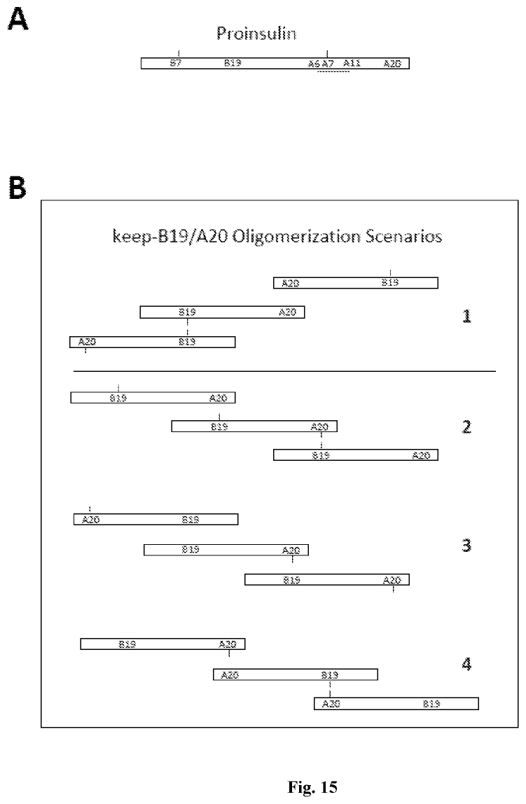

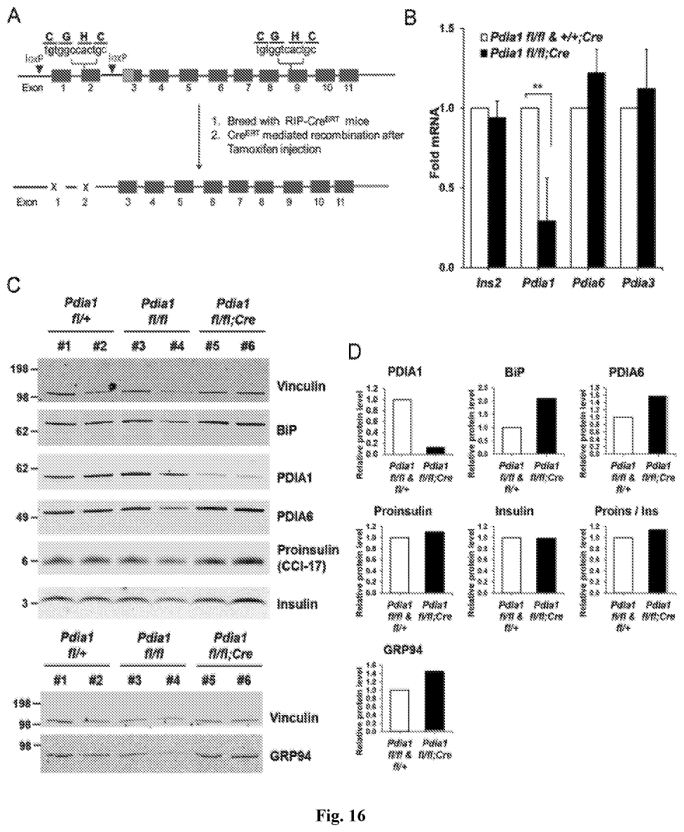

In these experiments, the inventors, surprisingly, noticed that 1) that all 2-Cys proinsulin mutants have the capability to at least weakly form a covalent dimer, which is essential to the further propagation of these improperly folded complexes into trimers, tetramers, and higher order complexes (FIG. 2B), and 2) a small fraction of proinsulin dimers appeared to remain even after reducing SDS-PAGE (FIG. 4D; FIG. 9). To understand these behaviors, the inventors expressed a series of mutant human proinsulins that retain only a single cysteine (and thus cannot make more than one intermolecular disulfide bond). In the experiment shown in FIG. 7A, 293T cells were transfected to individually express six distinct human proinsulin mutants bearing only one cysteine. At 28 h post-transfection, cell lysates were analyzed either by nonreducing SDS-PAGE (left) or incubated in SDS-gel sample buffer plus 200 mM DTT at room temperature for 10 min prior to SDS-PAGE. The gels were identically electrotransferred to nitrocellulose, and immunoblotted for proinsulin as in FIG. 6. FIG. 7B shows that seven independent experiments like that shown in the left panel of FIG. 7A were quantified for monomer to covalent dimer ratio: keep-A7 was mostly monomeric whereas keep-B19 predominantly formed covalent dimers (asterisk signifies p<0.05 when compared to each keep mutant except keep-A20). Although five of the six Cys residues could make a homotypic covalent association, Cys(A7) preferred not to make the disulfide bond (FIG. 7A left panel; quantified in FIG. 7B). More significantly, Cys(B19) not only covalently homodimerized with exuberance (FIG. 7A left panel) but this Cys(B19)-Cys(B19) bond could not be broken at room temperature in SDS-gel sample buffer even in the presence of 200 mM dithiothreitol (FIG. 7A right panel). These data indicate that a Cys(B19)-Cys(B19) disulfide bond between proinsulin monomers predisposes to strongly-associated covalent complexes that are automatically misfolded by omitting the crucial intramolecular Cys(B19)-Cys(A20) disulfide bond, which leaves the critical reactive Cys(A20) residue unpaired and available for further disulfide infidelity. Such an initiating event can thus form a nidus for assembly of larger misfolded disulfide-linked proinsulin complexes (FIG. 15).

It is recognized from human genome-wide association studies and animal models that progression from insulin resistance to pancreatic β-cell dysfunction is a linchpin in the development of T2D. Pancreatic β-cell ER stress is one of the most frequently described components of T2D in humans and animal models. While protein misfolding caused by mutations (in so-called conformational diseases) is one recognized cause of ER stress, the proximal trigger of beta cell ER stress early in the progression of T2D is unknown, but has been the subject of much speculation. It is indisputable that β-cell ER stress can be triggered by proinsulin misfolding in the setting of INS gene coding sequence mutations but there are also strong reasons to think that even in the absence of INS gene mutations, proinsulin misfolding could be an early feature in the progression of T2D.

The inventors have exploited several independent lines of experimentation to identify a significantly increased population of improperly folded proinsulin in the ER of prediabetic and diabetic β-cells. The inventors established that proinsulin misfolding includes the inability to successfully complete its three internal disulfide bonds, with a sub-fraction of proinsulin molecules in the ER bearing unpaired cysteine residues. This population of improperly folded proinsulin molecules enters into aberrant disulfide-linked partnerships with other proinsulin molecules in the ER, resulting in misfolded proinsulin complexes that are for the first time identified in the islets of human beings.

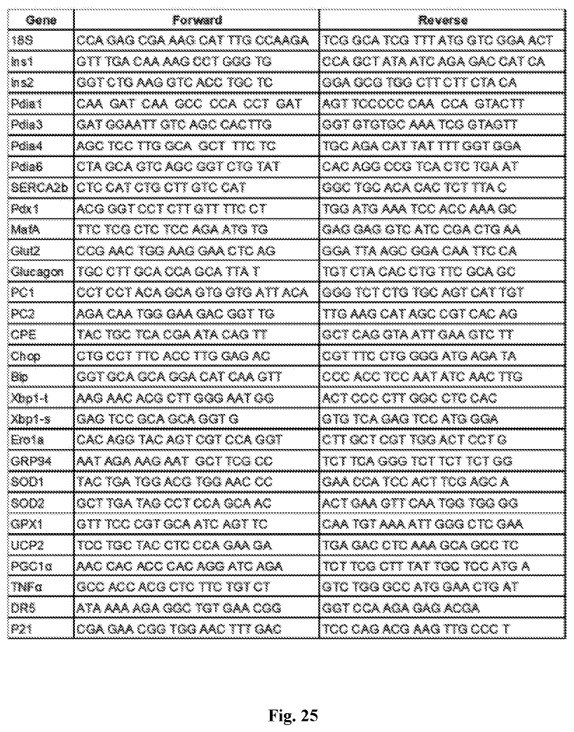

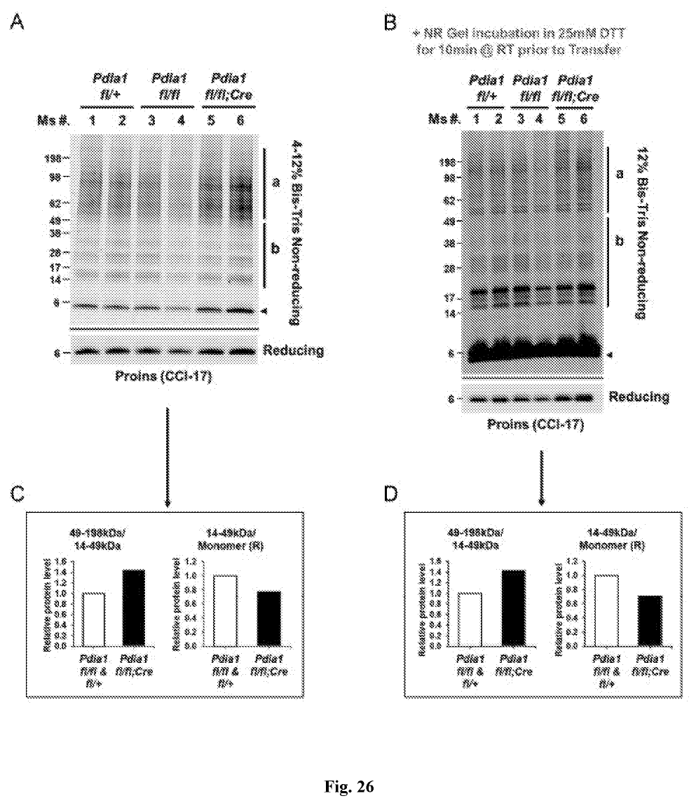

It is strongly suggested proinsulin disulfide-linked complex formation provides a status report on the health of the β-cell ER folding environment. Many studies point to the idea that the activity of ER stress (UPR) sensor proteins are required to actively maintain a proper ER chaperone and oxidoreductase environment for optimal proinsulin folding. As shown in the Example II, protein disulfide isomerase is one of the ER luminal factors that regulates the balance of proinsulin disulfide-linked complexes and monomers. Additionally, BiP plays a central role in ER stress sensing. In this Example, it is shown that even a modest decrease of BiP levels results in improperly folded proinsulin rapidly accumulating in disulfide-linked complexes (FIG. 25), supporting that proinsulin folding is highly sensitive to changes in the ER folding environment, which can include excessive proinsulin biosynthesis, altered chaperone and/or oxidoreductase expression, and changes in the rate of clearance of misfolded proinsulin molecules. It is contemplated that these and other ER luminal factors are needed to enhance the efficiency of formation of the proinsulin intramolecular Cys(B19)-Cys(A20) disulfide bond. Not only is this internal disulfide critical to additional native proinsulin disulfide pairing (FIG. 15), but the availability of free Cys(B19) and Cys (A20) can recapitulate the entire ladder of improperly disulfide linked proinsulin complexes. Several pathways of disulfide propagation are possible, for example, possible intermolecular disulfide-linked oligomerization involving homotypic B19-B19 and A20-A20 interactions (scenario 1), or heterotypic B19-A20 interactions (scenario 2), or combinations of homotypic and heterotypic interactions (scenarios 3+4) as shown in FIG. 15. Terminal Cys residues of disulfide-linked proinsulin oligomers exist as free thiols that can propagate into larger disulfide linked complexes, or could form mixed disulfides with ER oxidoreductases. However, those complexes bearing a homotypic Cys(B19)-Cys(B19) covalent bond are likely to be the most difficult to successfully isomerize, as this bond cannot be broken in vitro even in the presence of SDS plus 200 mM DTT (FIG. 7A).

A specific goal of T2D research is to better understand the natural history of β-cell failure. What has recently emerged using the LepRdb/db mouse model, is that beginning around 4-5 months of life, after hyperglycemia has appeared, β-cells appear to be in the early stages of dedifferentiation, and with a similar time course, β-cell apoptosis may occur in parallel. However, clearly there are important islet changes that occur considerably before these events including β-cell ER stress response activation. FIG. 8 presents a schematic describing the hypothesis regarding how proinsulin misfolding fits into the paradigm of what is known about T2D progression in the LepRdb/db model. In detail, A schematic is shown, indicating progression of early islet dysfunction during the natural history of diabetes in the LepRdb/db mouse. In the first stage of postnatal life, random blood glucose is in the normal range and insulin content in islets is actually slightly greater than that seen in the control condition. Total proinsulin levels are not diminished, but there is a slight increase in proinsulin disulfide-linked complexes. At the second stage, random hyperglycemia (150-300 mg/dL) is observed, accompanied by islet insulin levels that are less than that seen in the control condition. However, total proinsulin levels are notably increased, accompanied by an increase in proinsulin disulfide-linked complexes. At a third stage is a worsening of random hyperglycemia (>450 mg/dL), accompanied by low islet insulin and low islet proinsulin, which has been attributed to β-cell dedifferentiation, or β-cell death, or could potentially represent a combination of both. This Example highlights that throughout the first, compensated stage, improperly folded proinsulin is increased but islet insulin content is still robust (FIGS. 4D, 5C). In the second, decompensating stage, islet proinsulin levels are actually further increased, but much of this is improperly folded proinsulin in the ER, and at this stage, islet insulin levels begin to drop (FIG. 5). It is at a still higher level of hyperglycemia that both proinsulin and insulin steady state levels are low (FIG. 8), which correlates with the time when β cell dedifferentiation and β cell death have been reported. It should be noted that even at this time, the low-level proinsulin protein that is still expressed is recovered >90% in aberrant disulfide linked complexes, and even partial restoration of secretory pathway function requires β-cell rest.

In conclusion, the data in this Example establishes the presence of a previously underappreciated population of aberrant proinsulin complexes that accumulates in prediabetic conditions and persists until β cell failure ensues. Further, the data presented herein establish disulfide-linked complexes of proinsulin as one of the earliest tissue biomarkers indicating β-cell secretory pathway dysfunction, which is associated with ER stress and ultimate insulin deficiency that occurs in the natural progression of T2D.

### Example II: PDIA1/P4HB is Required for Efficient Proinsulin Maturation and β Cell Health in Response to Diet Induced Obesity

Mice: C57BL/6 mice with Pdia1 foxed alleles were obtained from Dr. J. Cho (Univ. of Illinois-Chicago) and crossed with Rat Insulin Promoter (RIP-Cre^{ERT}) transgenic mice. Congenic Pdia1 gene floxed littermates with or without the Cre^{ERT} transgene were used for in vivo experiments. Pdia1 deletion was performed by injection of the estrogen receptor antagonist Tamoxifen (Tam) (4 mg/mouse) three times a week. Male mice were pair-housed for the high fat diet (HFD) study.

Generation and culture of primary mouse embryonic fibroblast (MEF): WT C57BL/6 d14 embryos were isolated under sterile conditions and placed in 100 mm cell culture dish containing PBS. Placental and other maternal tissues were removed and the embryos were washed three times with PBS. Heads and visceral organs were removed. The heads were used for genotyping. Embryos were finely minced with a sterile razor blade and treated with 1 ml of 0.25% trypsin/EDTA (Corning) for 40 min at 37° C. DMEM medium (Corning) supplemented with 10% FBS, 1% penicillin/streptomycin, 100 μg/ml primocin and 1 mM sodium pyruvate was added to quench trypsin activity. Tissue homogenate was subsequently disaggregated by repeated pipetting, and further centrifuged to collect the fibroblast cell pallet. Cells were plated in 100 mm culture dish in DMEM medium at 37° C. in a cell culture incubator under 5% CO₂. To prevent mycoplasma, bacterial, and fungal contamination, primary MEFs were cultured in medium containing 100 μg/ml primocin (InvivoGen) and used for experiments prior to passage #5.

Glucose and Insulin Tolerance Tests: Glucose tolerance tests were performed by IP injection of glucose (1g/Kg body weight) into mice after fasting for 4 h. For insulin tolerance tests, 1.5 units/Kg of insulin was injected IP into mice after a 4 h fast. Blood glucose levels were measured by tail bleeding at each time point indicated.

Measurement of Serum Proinsulin and Insulin: Mice were fasted O/N and re-fed for 4 h. Blood was collected by retro-orbital bleeding and serum was prepared by centrifugation. Serum proinsulin and insulin levels were measured by ELISA (Mercodia, 10-1232-01, 10-1247-01) according to the manufacturer's protocol.

Islet Isolation: Islets were isolated by collagenase P (Roche) perfusion as described following by histopaque-1077 (Sigma-Aldrich, Inc. St. Louis) gradient purification. Islets were handpicked and studied directly or after overnight culture in RPMI 1640 medium (Corning 10-040-CV) supplemented with 10% FBS, 1% penicillin/streptomycin, 100 μg/m1 primocin, 10mM Hepes, and 1mM sodium pyruvate.

Islet RNA Isolation and qRT-PCR: Total RNAs were extracted from isolated islets by RiboZol™ Extraction reagent (VWR Life Science). cDNA was synthesized by iScript™ cDNA Synthesis kit (Bio-Rad Laboratories, Inc.). The relative mRNA levels were measured by qRT-PCR with iTaq™ Universal SYBR green Supermix (Bio-Rad Laboratories, Inc.). All primers are listed in FIG. 25.

Islet Western Blotting: Isolated islets were lysed in RIPA buffer (10 mM Tris pH 7.4, 150 mM NaCl, 0.1% SDS, 1% NP-40, 2mM EDTA) with protease and phosphatase inhibitors (Fisher Scientific) on ice for 10 min and lysates were collected after centrifugation at 4° C. for 10 min at 12000 g. Samples were prepared in Laemmli sample buffer without (non-reducing) or with (reducing) 5% β-mercaptoethanol. After boiling for 5 min, samples were analyzed by SDS-PAGE (4-12% Bis-Tris gel, (Bio-Rad Laboratories, Inc.)) and transferred to nitrocellulose membranes (Bio-Rad Laboratories, Inc.). For DTT incubation prior to transfer, non-reduced samples were electrophoresed on a 12% Bis-Tris SDS gel and then incubated in 25 mM DTT for 10 min at RT. Primary antibodies were as follows: α-vinculin (Proteintech, 66305-1-1g), α-GRP94 (Cell Signaling, 20292P), α-BiP (BD Biosciences, 610979), α-PDIA1 (Proteintech, 11245-1-AP), α-PDIA4 (Proteintech, 14712-1-AP) α-PDIA6 (Proteintech, 18233-1-AP), α-proinsulin (HyTest Ltd., 2PR8, CCI-17). Guinea pig polyclonal α-insulin antibody was produced in-house. For secondary antibodies, goat α-mouse, goat α-rabbit, and donkey α-guinea pig antibodies were used in 1:5000 (Li-Cor, IRDye®-800CW or IRDye®-680RD).

Pancreas Tissue Transmission Electron Microscopy: Samples were prepared according to the UCSD Cellular & Molecular Medicine Electron Microscopy Facility protocols. Mouse pancreas were perfused in modified Karnovsky's fixative (2.5% glutaraldehyde and 2% paraformaldehyde (PFA) in 0.15M sodium cacodylate buffer, pH 7.4) and fixed for at least 4 h, post-fixed in 1% osmium tetroxide in 0.15M cacodylate buffer for 1 h and stained en bloc in 2% uranyl acetate for 1h. Samples were dehydrated in ethanol, embedded in Durcupan epoxy resin (Sigma-Aldrich, Inc. St. Louis), sectioned at 50 to 60 nm on a Leica UCT ultramicrotome, and delivered to Formvar and carbon-coated copper grids. Sections were stained with 2% uranyl acetate for 5 min and Sato's lead stain for 1 min. Images were obtained using a Tecnai G2 Spirit BioTWIN transmission electron microscope equipped with an Eagle 4k HS digital camera (FEI, Hilsboro, Oreg.) with indicated magnifications. Insulin granule numbers were counted manually on images taken at 4800× magnification and divided by islet area measured by image J software. Insulin mature granule vesicles and dense core sizes were also measured by image J software.

Pancreas Immunohistochemistry: Pancreata were harvested and fixed in 4% PFA. Paraffin embedding, sectioning, and slide preparations were done in the SBP Histopathology Core Facility. Sections were stained with the following antibodies; α-glucagon (Abcam, K79bB10), α-PDIA1 (Proteintech, 11245-1-AP), α-proinsulin (HyTest Ltd., 2PR8, CCI-17), and DAPI (Fisher Scientific). Guinea pig polyclonal α-insulin antibody was produced in-house. For secondary antibodies, Alexa Fluor® 488 goat α-rabbit IgG, Alexa Fluor® 488 goat α-mouse IgG, Alexa Fluor® 594 goat α-mouse IgG, and Alexa Fluor® 594 goat α-guinea pig IgG antibodies were used (Invitrogen). Images were taken by Zeiss LSM 710 confocal microscope with a 40× objective lens. Scale bar, 20 μm. For β cell area measurement, pancreata were harvested, fixed in 4% PFA and embedded in paraffin. Three sections were prepared at 200 μm intervals for each pancreas and stained with guinea pig polyclonal insulin antibody and DAPI. Alexa Fluor® 594 goat α-guinea pig IgG was used as a secondary antibody. Images were taken by an Aperio FL Scanner (Leica). Insulin stained β cell area, islet area, and pancreas area were measured by Aperio Imagescope software.

Analysis of Oxidative Stress: Islets were plated onto CellCarrier™-96 ultra microplates (Perkin Elmer) in phenol red-free RPMI 1640 medium supplemented with 10% FBS, 1% penicillin/streptomycin, 100 μg/ml primocin, 10mM Hepes, and 1 mM sodium pyruvate one day prior to staining. Islets were treated with menadione (AdipoGen Life Science, 10 μM) for 3 h at 37° C. Islets were stained with CellROX® Deep Red reagent (Molecular Probes, C10422, 100 μM) and Hoechst 33342 (Invitrogen, 10 μg/ml) for 1 h at the same time. Islets were washed three times with HBSS and incubated in HBSS for imaging while temperature and CO₂ were controlled. Images were obtained by an Opera Phenix high content screening system (63× objective lens) in the SBP High Content Screening (HCS) Facility and seven z-stack images (1 μm interval) were combined.

Statistical Analysis: Data are indicated as Mean±SEM. Statistical significance was evaluated by unpaired two-tailed Student's t test. P-values are presented as *; P<0.05, **; P<0.01, ***; P<0.001.

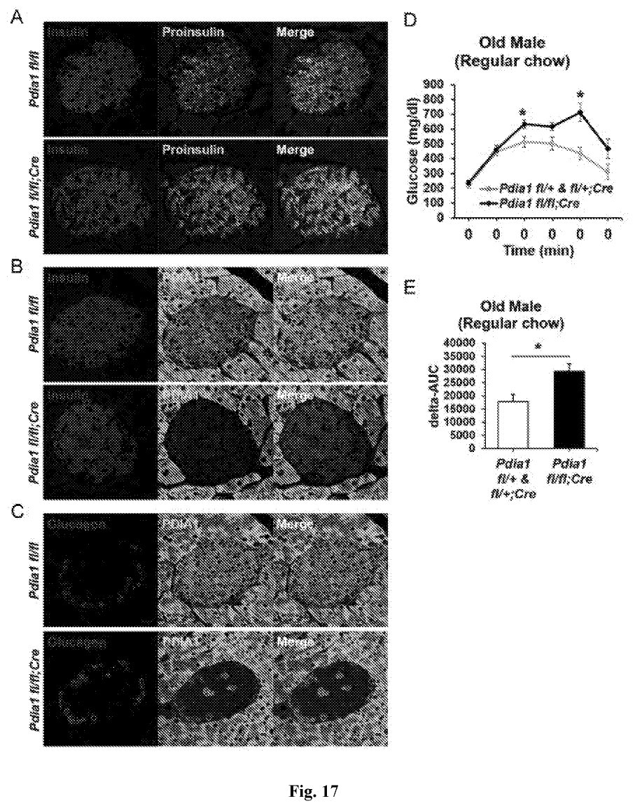

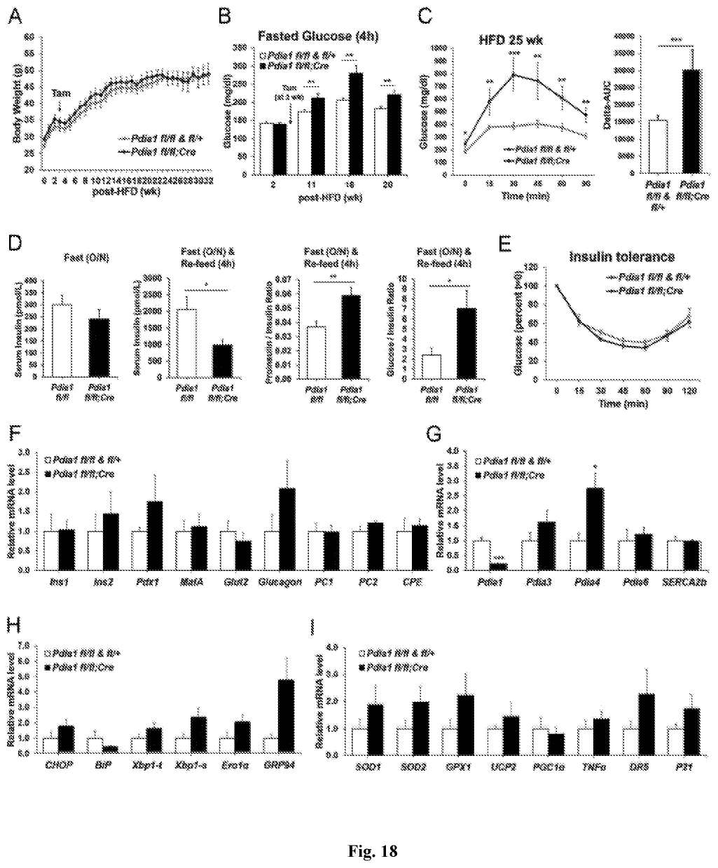

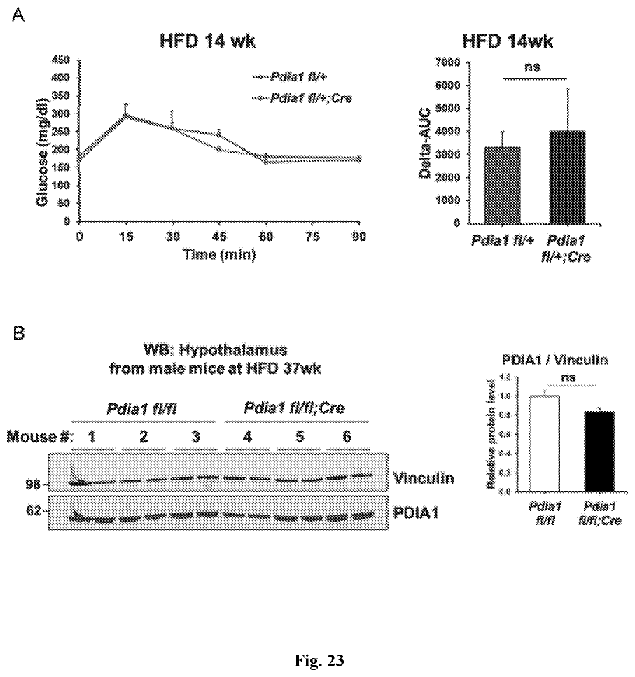

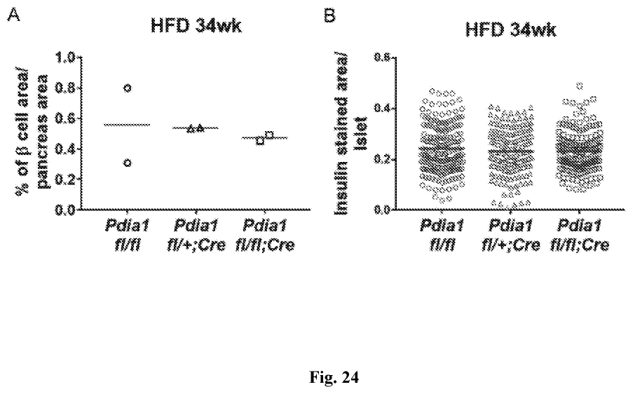

Results: As PDIA1 is highly expressed in islets, the inventors analyzed β cell-specific conditional Pdia1-null mice using tamoxifen (Tam)-regulated deletion of floxed Pdia1 alleles through rat insulin promoter driven Cre-recombinase (RIP-Cre^{ERT}). Quantitative RT-PCR (qRT-PCR) demonstrated an ˜75% decrease in Pdia1 mRNA in isolated islets from the β cell-specific Pdia1-knock out mice (Pdia1 fl/fl;Cre^{ERT} herein, KO, but genotypes with no effects on Insulin 2, Pdia6 or Pdia3 mRNAs (FIG. 16). Early studies demonstrated that mice with or without the RIP-Cre^{ERT} allele did not show significant differences in glucose homeostasis after a 14 wk HFD (FIG. 23). Therefore, the inventors compared mice with two floxed alleles (fl/fl) and mice with one foxed and one wildtype (WT) allele (fl/+) with littermates that also harbor the RIP-Cre^{ERT} transgene, both before and/or after Tam injection. Western blotting of isolated islets from Tam-treated mice with the RIP-Cre^{ERT} allele demonstrated significantly reduced PDIA1 protein with increased expression of the UPR genes BiP, PDIA6 and GRP94 (FIG. 16C-D), suggesting Pdia1 deletion may cause ER stress in β cells of the KO mice. Immunohistochemistry (IHC) of pancreas tissue sections with antibodies specific for proinsulin/insulin, glucagon and PDIA1 confirmed the absence of PDIA1 in a β cell-specific manner in the KO mice (FIG. 17A-B, compare middle panels). Interestingly, analysis of glucagon staining in pancreas sections detected very low PDIA1 expression in islet a cells (FIG. 17C, middle panels), and it is noted that proglucagon contains no disulfide bonds.

In more detail, FIG. 16A shows a diagram depicts the generation of Pdia1:RIP-Cre^{ERT} mice. Mice with foxed Pdia1 alleles were crossed with RIP-Cre^{ERT} transgenic mice and progeny were injected IP with Tam to induce Cre^{ERT} function and Pdia1 deletion. Control littermate mice with one or two floxed Pdia1 alleles, but not harboring the RIP-Cre^{ERT} transgene, were injected in parallel with Tam. In the experiment shown in FIG. 16B, total RNA was extracted from islets isolated from female mice at 8 wks after Tam injection. mRNA levels were measured by qRT-PCR. Mean±SEM, n=3 for each group (P<0.01**). In the experiment shown in FIG. 16C, western blot illustrates expression of Vinculin, PDIA1, BiP, PDIA6, GRP94, Proinsulin, and Insulin in islets isolated from female mice at 14 wks after Tam injection. In FIG. 16D, quantification of indicated proteins by Western blotting (from FIG. 16C) is shown. Each value was normalized to vinculin except for the proinsulin to insulin ratio. Pdia1 fl/fl (n=2), Pdia1 fl/+ (n=2), Pdia1 fl/fl;Cre (n=2).

FIGS. 17A-E show data indicating that Pdia1 is specifically and persistently deleted in murine β cells. In FIGS. 17A-C, pancreas tissue sections were prepared from female mice at 49 wks after Tam injection and immuno-stained with anti-proinsulin, insulin, PDIA1, and glucagon antibodies. Images were merged with DAPI stain. Scale bar, 20 μm. In FIG. 17D, old KO mice developed glucose intolerance compared to control genotypes measured by glucose tolerance testing (GTT) at 9 wks after Tam injection. Male mice at 9 month of age were injected with Tam and fed a regular chow. Mice were fasted (4 h) prior to IP glucose injection (2 g/Kg body weight) and glucose levels were measured by tail bleeding at each time point (0; non-injected, 15, 30, 60, 90 min). Control genotypes: Pdia1 fl/+(n=2), Pdia1 fl/+;Cre (n=5), KO; n=5. In FIG. 17E, area under the GTT curve (Δ-AUC) of (FIG. 17D) is indicated in graph.

Although the RIP-Cre^{ERT} allele was reported to be expressed in the hypothalamus, Western blotting of PDIA1 in hypothalamic tissue did not detect reduced PDIA1 expression (FIG. 23). FIGS. 23A-B show that the RIP-Cre^{ERT} allele does not impact the β cell-specific Pdia1 deletion phenotype. In FIG. 23A, Pdia1 fl/+ mice harboring the RIP-Cre^{ERT} allele (n=3) exhibit similar glucose tolerance tests as Pdia1 fl/+mice without the RIP-Cre^{ERT} allele (n=4) at 14 wks after HFD. All mice were injected with Tam after 3 wks of HFD. In FIG. 23B, Δ-AUC of glucose tolerance test in FIG. 23A. In FIG. 23C, PDIA1 protein levels did not change in hypothalamic brain tissue after 37 wks of HFD. Pdia1 fl/fl (n=3), Pdia1 fl/fl;RIP-Cre (n=3). All data are shown as Mean±SEM. In addition, this RIP-Cre^{ERT} allele did not affect serum dopamine, which is synthesized in the arcuate nucleus of the hypothalamus. Although Cre positive staining was detected in the hypothalamus, there was no difference in expression of growth hormone-releasing hormone (GHRH) in the KO mice compared to the control genotypes with the RIP-Cre^{ERT} allele.

Young male and female mice did not exhibit any defects in glucose homeostasis. However, on a regular chow diet, the challenge of aging in KO males caused statistically significant increase in glucose intolerance (as shown in FIG. 17).

KO mice Fed a High Fat Diet (HFD) Become Glucose Intolerant with Defective Insulin Production.

To determine the role of β cell PDIA1 in the face of metabolic stress, male mice were fed a 45% HFD. All mice were Tam injected at 3 wks after HFD was started. Genetic controls (Pdia1 fl/fl and fl/+) and KO mice fed HFD up to 32 wks showed no significant differences in body weight or weight gain (FIG. 18A). However, fasting blood glucose (4 h) in the HFD mice was significantly elevated in the KO versus genetic controls (Fasting (4 h) blood glucose levels were elevated in KO mice at 11, 16 and 20 wks after HFD, FIG. 18B). In addition, glucose intolerance was observed in HFD-fed KO mice as measured by the difference in the area under the glucose excursion curve (Delta-AUC) (FIG. 18C). In detail, KO mice displayed higher blood glucose levels and area under the GTT curve (Δ-AUC) compared to control genotypes during glucose tolerance testing (GTT) after HFD for 25 wks. GTT were performed at multiple time points after HFD in two independent cohorts and representative results are shown. Mice were fasted (4 h) prior to IP glucose injection (1g/Kg body weight) and glucose levels were measured by tail bleeding at each time point (0; non-injected, 15, 30, 60, 90 min). Control genotypes; n=17, KO; n=6. Serum insulin levels were decreased in HFD-fed KO mice, and a fasting-refeeding challenge revealed hypoinsulinemia with an increased proinsulin/insulin ratio (FIG. 18D). Pdia1 deletion did not affect insulin sensitivity measured by insulin tolerance tests (FIG. 18E) and no significant difference was observed in the percent of β cell area to total pancreas or β cell area per islet in HFD-fed KO mice (FIG. 25). These results indicate that in the setting of metabolic challenge, Pdia1 is required for adequate insulin production to maintain systemic glucose homeostasis.

Pdia1 is not Required for Expression of β Cell-Specific Genes, Antioxidant Response Genes or Cell Death Genes.

To reveal whether Pdia1 deletion impacts gene expression to cause β cell dysfunction, islets from male mice after 30 wks of HFD were isolated and analyzed mRNA levels by qRT-PCR. The results demonstrated no significant decrease in β cell- and a cell-specific mRNAs, mRNAs encoding the insulin processing enzymes PC1/3, PC2, and CPE, or mRNAs encoding β cell transcription factors PDX1 or MAFA (FIG. 18F). Therefore, the reduced insulin content in KO serum and islets (FIG. 18D) was not due to reduced β cell-specific gene expression. There was also no significant change in expression of other PDI family members and Serca2b, except for Pdia4 (FIG. 18G). In addition, UPR genes were not significantly elevated at this point in time in the Pdia1 KO islets, other than Grp94 (FIG. 18H), which correlated with a slight increase in protein (FIG. 16). Lastly, there were no significant differences in expression of a panel of genes representing the antioxidant response and cell death (FIG. 181). These results show that β cell-specific Pdia1 deletion does not alter expression of β cell specific genes, antioxidant response genes, or cell death genes.

β Cell Pdia1 KO Mice Fed HFD Exhibit β Cell Failure with Distinct Morphological Aberrations.

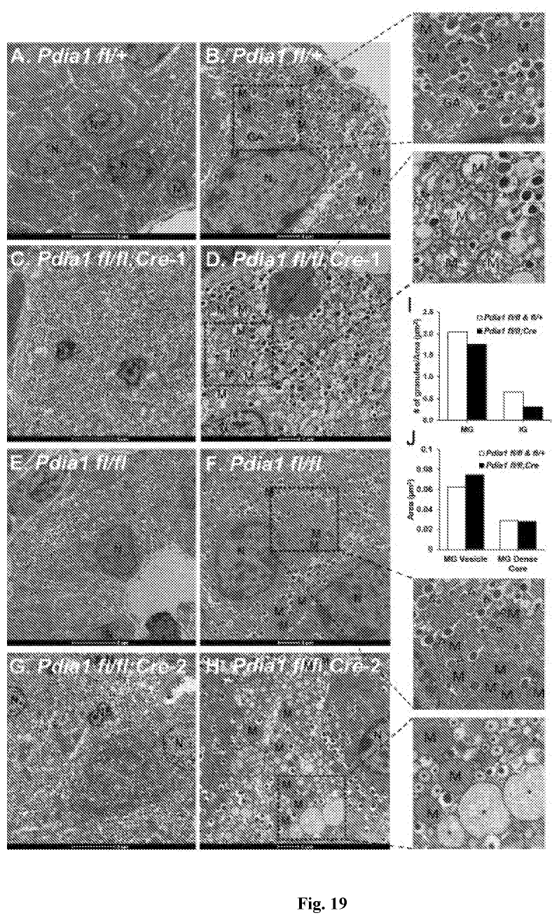

The impact of Pdia1 deletion on β cell ultrastructure was analyzed by transmission electron microscopy (TEM) (FIG. 19). TEM did not detect any significant morphological alterations in the cohorts of KO mice fed a regular diet for 10 wks after Tam injection (data not shown). Strikingly however, β cells from HFD-fed KO mice showed significant abnormalities including ER vesiculation and distension, mitochondrial swelling, and nuclear condensation, that were not observed in genetic control mice (Pdia1 and fl/+) (FIG. 19). The red asterisks represent significantly distended ER, reflecting ER stress (FIG. 19D,H, expanded). To determine whether Pdia1 deletion also affects insulin granule content, the number of mature (dense dark core, yellow arrows in enlarged image) and immature (inner gray core, orange filled open arrowheads in enlarged image) granules was quantified and discovered that KO mice had fewer mature (˜15%) and immature (˜50%) granules compared to the control genotypes fl/fl and fl/+ (FIG. 19I). Analysis of the cross-sectioned mature granule (MG) vesicle area and dense core size demonstrated that the average cross-sectional area of MGs in KO mice was 20% larger than control genotypes (FIG. 19J), however, there was no difference in MG dense core size between genotypes (FIG. 19J), suggesting that insulin packaging efficiency within granules was decreased in Pdia1 deleted-β cells. This is consistent with decreased serum insulin levels in metabolically-challenged KO mice (FIG. 18). Taken together, the findings indicate that Pdia1 is essential to maintain β cell ultrastructure upon metabolic stress, indicative of suboptimal β cell function.

KO Islets Have an Increased Intracellular Proinsulin/Insulin Ratio with Accumulation of High Molecular Weight (HMW) Proinsulin Complexes.

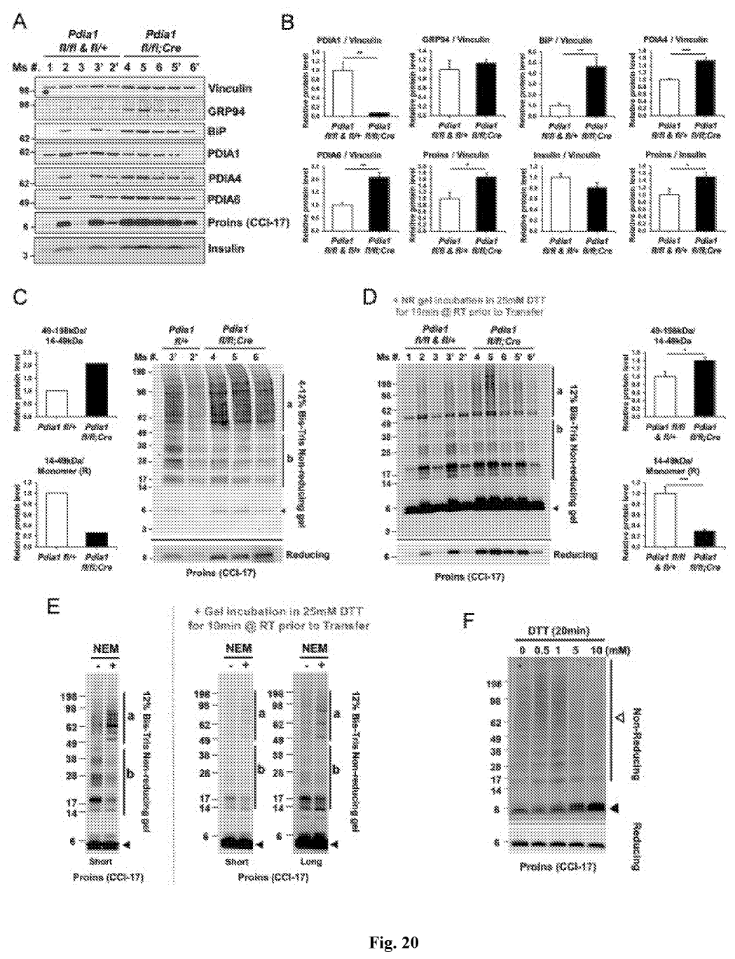

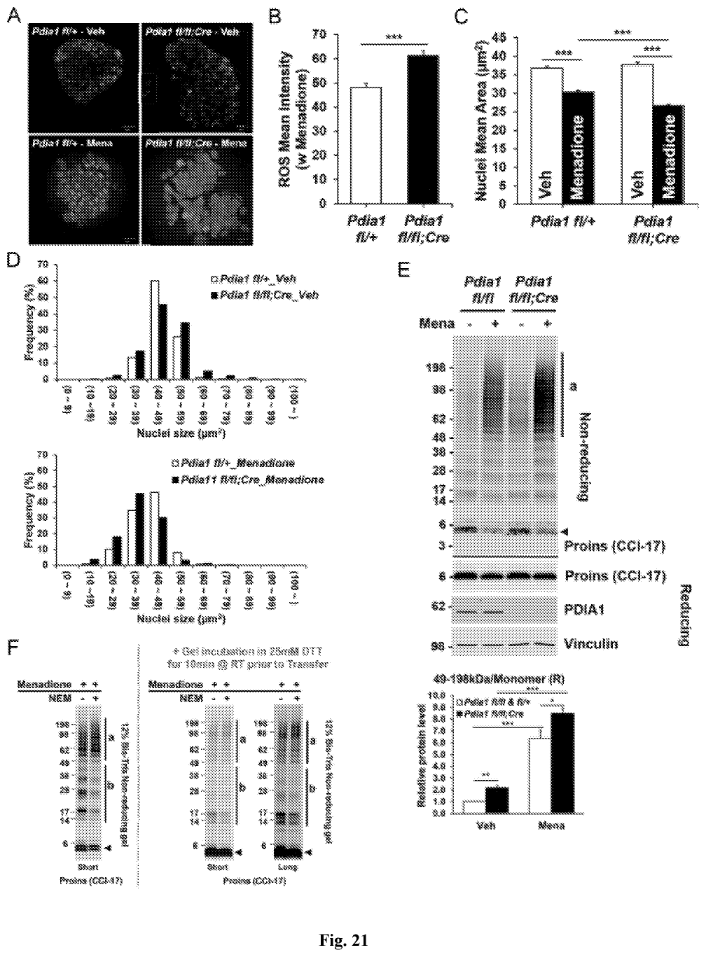

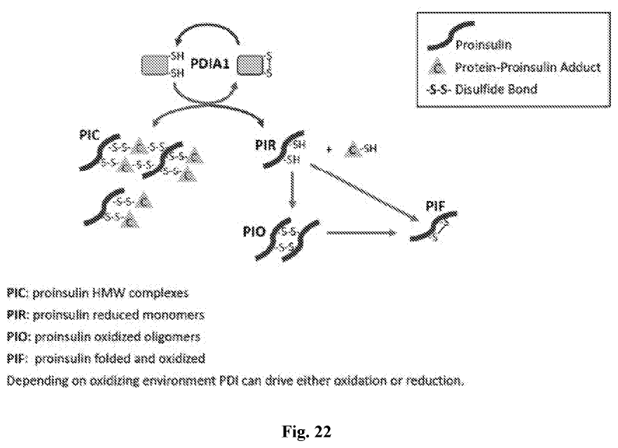

The impact of Pdia1 deletion on proinsulin folding was investigated by analysis of proinsulin and insulin steady state levels in islets isolated from male mice after 30 wks of HFD using Western blotting under reducing and non-reducing conditions. The level of PDIA1 protein was decreased in islets isolated from KO mice, as expected (FIG. 20A-B). Insulin levels were lower in KO islets compared to control genotypes while the proinsulin/insulin ratio was significantly elevated (FIG. 20A-B). BiP/Hspa5 was induced with increased PDIA4 and PDIA6 in Pdia1-deleted versus Pdia1-sufficient islets (FIG. 20A-B). The increased levels of BiP are consistent with the notion that the absence of Pdia1 causes a mild ER stress, suggestive of protein misfolding, which is also consistent with ER distension (FIG. 19).

The potential role of PDIA1 in disulfide maturation encouraged us to look for the presence of proinsulin disulfide-linked complexes by non-reducing SDS-PAGE (FIG. 20C). Western blot analysis under non-reducing conditions using the CCI-17 antibody demonstrated the appearance of multiple proinsulin bands including a monomer at ˜6kDa (black arrowhead, FIG. 20C) and a ladder of molecular masses that could correspond to proinsulin dimers up to pentamers (b, 14-49 kDa) as well as a cluster of HMW complexes (a, 49-198 kDa) in Pdia1 fl/fl and KO islets (FIG. 20C). Pdia1 deletion increased the amount of HMW complexes relative to the oligomeric forms and decreased the oligomers relative to monomeric proinsulin compared to the genetic controls without the RIP-Cre^{ERT} allele (FIG. 20C, quantified in graph).

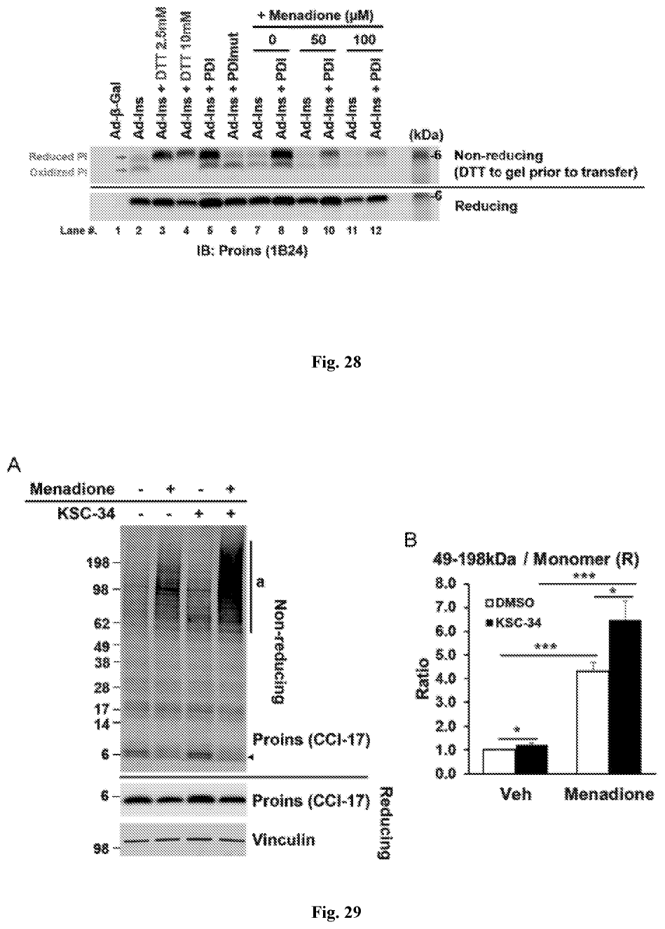

In this Example, the inventors discovered that the epitope reactivity of the CCI-17 monoclonal antibody is very dependent the status of proinsulin sulfhydryls and disulfide bonds. Two major findings support this conclusion. First, when the non-reducing SDS-PAGE gel was treated with dithiothreitol (DTT) prior to transfer to nitrocellulose, the inventors observed a great increase in antibody reactivity with monomeric proinsulin (FIG. 20C-D). This suggests that opening the proinsulin molecule significantly enhances epitope exposure to the CCI-17 antibody. It is also important to note that western blotting analysis of the non-reducing SDS-PAGE gel (with or without subsequent DTT treatment of the gel prior to transfer) demonstrated readily detectable proinsulin disulfide-linked complexes in WT murine islets, nondiabetic human islets and in prediabetic db/db islets prior to onset of hyperglycemia. The second finding is that treatment with N-ethylmaleimide (NEM) to alkylate free sulfhydryls slightly increased the HMW proinsulin-containing complexes and slightly reduced the disulfide-linked oligomers (FIG. 20E). Such results implicates that PDIA1 may be required to reduce the HMW complexes and NEM treatment inactivates PDIA1, thereby stabilizing the HMW complexes. Similar results were obtained with a selective PDIA1 inhibitor (FIG. 28)

To gain further insight into the nature of the disulfide-linked oligomers, islets isolated from WT male mice fed a regular diet were treated in culture with increasing concentrations of dithiothreitol (DTT) to increase the ER reduction potential. This treatment produced increasing amounts of the proinsulin monomer and residual disulfide-linked dimer (FIG. 20F). In addition, female KO mice fed a regular diet for 14 wks after Tam also showed increased HMW complexes relative to the disulfide-linked proinsulin oligomers and reduced oligomers relative to monomeric proinsulin compared to the genetic controls.

Oxidant Treatment of Pdia1 KO Islets Increases Accumulation of HMW Proinsulin Complexes.

Because ER stress is linked with oxidative stress, the inventors tested whether Pdia1 deletion confers increased sensitivity to oxidants by treating islets (isolated after 30 wks of HFD) with the vitamin K analogue menadione, which can render cells susceptible to apoptosis by increasing cytosolic calcium with nuclear condensation. As shown in FIG. 21A, islets isolated from mice after 30 wks HFD were treated with or without Menadione (10 μM, 3 h) and co-stained with CellROX Deep Red (red) and Hoechst 33342 (blue). Live islet images were obtained by an Opera Phenix high content screening system (63× objective lens) and seven z-stack images (1 μm interval) were combined. Scale bar, 20 μm. genetic controls; n=3, KO; n=3. In FIG. 21B, quantification of ROS mean intensity is shown. CellROX Deep Red mean intensity (divided by area) was measured by image J software. Mean±SEM, P<0.001***. In FIG. 21C, quantification of nuclear mean area (μm²) measured in Hoechst 33342 stained images by ImageJ software is shown. Mean±SEM, P<0.001***. In FIG. 21D, Histogram analysis of nuclear sizes is shown. Percent frequencies are indicated in the graph. In FIG. 21E, western blot of islets isolated from mice after 37 wks of HFD by SDS-PAGE under reducing and non-reducing conditions is shown. After overnight recovery, islets were treated with menadione (100 μM) for 1 h. Islet preparations from five independent control and KO mice were performed and representative images are shown. Quantification of the ratio of HMW proinsulin complexes (a) to monomeric proinsulin under reducing conditions is shown in graph (lower). Mean±SEM, P<0.05*, P<0.01**, P<0.001***. Controls; n=5, KO; n=5 mice. In FIG. 21F, WT murine islets were treated with Menadione (100 μM) for 1h, treated with or without NEM as in FIG. 20E, and lysates were prepared and analyzed under non-reducing conditions.

Menadione treatment significantly increased ROS as observed by CellROX Deep Red stain (FIG. 21A) and β cells with Pdia1 deletion showed greater ROS accumulation than those of genetic control fl/+ islets (FIG. 21B). Furthermore, both the average size and the histogram-analyzed nuclear size after Hoechst 33342 staining demonstrated that menadione promoted nuclear condensation (FIG. 21C) and this effect appeared to be greater in KO islets compared to the genetic controls (FIG. 21D). To uncover how Pdia1 deletion may affect the sensitivity of proinsulin maturation to perturbation by oxidants, western blotting of islet lysates was performed under reducing and non-reducing conditions. Consistently, in the absence of menadione treatment, Pdia1 deletion increased the HMW proinsulin complexes (a, 49-198 kDa) compared to WT islets, and this effect was even greater in islets treated with menadione (FIG. 21E). Importantly, DTT treatment of the gel prior to nitrocellulose transfer demonstrated no difference between untreated and NEM-treated islets (FIG. 6F). Taken together, these results show that β cells lacking Pdia1 are more sensitive to oxidizing conditions that promote the formation of HMW proinsulin complexes.

Proinsulin Accumulation in the ER Increases Oligomeric and HMW Complexes.

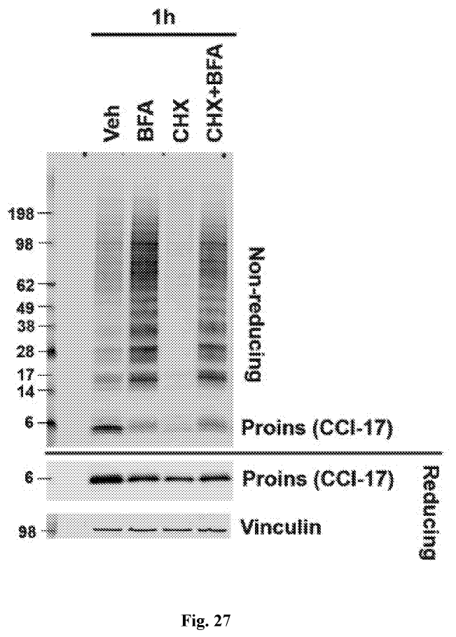

The experimental results in this Example show that PDIA1 is required to prevent formation of HMW proinsulin complexes. Also, it is contemplated that increased proinsulin synthesis predisposes to oligomer and HMW complex formation. To test the notion that increased proinsulin expression, as in T2D, may exacerbate abnormal disulfide formation, the effect of brefeldin A (BFA), which promotes retrograde COP1 trafficking from the cis-Golgi to the ER to prevent export of secretory proteins to the Golgi, and thus increasing their concentration in the ER, was tested. BFA treatment significantly increased proinsulin complex formation, thus supporting the notion that the aberrant multimers/HMW complexes are a consequence of increased proinsulin content in the ER (FIG. 27). Treatment with the translation elongation inhibitor cycloheximide (CHX), did not significantly affect the results, other than an expected reduced proinsulin content, indicating the presynthesized proinsulin is subject to multimer/HMW complex formation when its abundance in the ER is increased.

PDIA1 is a Reductase that Facilitates Proper Proinsulin Folding.

To study the impact of PDIA1 in the absence of C peptide processing in a more manipulatable system, the inventors analyzed human proinsulin expression delivered by adenovirus to murine embryonic fibroblasts (MEFs) that were co-infected with WT PDIA1 or PDIA1 with 4 Cys to Ser mutations in the two vicinal catalytic PDI sites. Infection with Ad-hProins produced significant amounts of reduced and oxidized proinsulin upon analysis by non-reducing SDS-PAGE with DTT treatment prior to transfer to nitrocellulose (FIG. 28). Treatment of cells with increasing concentrations of DTT increased the amount of reduced proinsulin (lanes 3,4), as expected. Significantly, forced expression of PDIA1, but not catalytically inactive PDIA1, increased the amount of reduced proinsulin (lanes 5, 6, 8, 10 and 12). Therefore, the results support the hypothesis that PDIA1 acts as a reductase to prevent aberrant proinsulin oxidation, consistent with previous studies in vitro.

Pharmacological Inhibition of PDIA1 Recapitulates Effects of Pdia1 Gene Deletion.

With recent development of a selective PDIA1 inhibitor that covalently interacts with the a catalytic motif in PDIA1, the effect of this inhibitor in murine WT islets as well as the impact of oxidant menadione treatment was tested. WT murine islets were treated with menadione in the presence or absence of 3004 KSC-34 for 3 h and then treated with menadione (100 μM) for 1 h at 37° C. Five independent experiments were performed and representative results are shown in FIG. 29. The results show that the KSC-34 inhibitor alone slightly increased HMW complexes. However, when combined with menadione, KSC-34 treatment recapitulated the effect of Pdia1 deletion upon menadione treatment (FIG. 29). Importantly, the findings demonstrate that either a PDIA1 chemical inhibitor or gene deletion exhibit similar effects on the generation of HMW complexes, especially upon oxidant treatment.

PDIA1 is the major ER oxidoreductase in the majority of mammalian cells, including β cells. Although several in vitro studies demonstrated that PDI actively engages proinsulin to catalyze disulfide bond formation, there is little information regarding the significance of PDI action in vivo. Here, using β cell specific Pdia1 deletion, it is shown that PDIA1 is increasingly important for insulin production in the face of either age or metabolic stress imposed by a HFD. Specifically, when compromised by HFD feeding, mice with β cell-specific Pdia1 deletion displayed exaggerated glucose intolerance with significant β cell abnormalities including diminished islet and serum insulin accompanied by an increased proinsulin/insulin ratio in islets and serum (FIG. 18), with diminished insulin packaging and storage in secretory granules and a reduced number of insulin secretory granules (FIG. 19). In addition, β cell Pdia1 deletion caused abnormal ultrastructural changes including ER distension and vesiculation, mitochondrial swelling, and nuclear condensation (FIG. 19). Pdia1-deleted islets were also sensitive to oxidant challenge (FIG. 21), which is significant because PDIA1 is a highly abundant ER protein (˜mM concentration) that is primarily in a reduced form and although it cycles, it may significantly contribute to redox homeostasis in the ER.

PDIA1 might assist proinsulin folding by facilitating proper intramolecular disulfide bond formation. Alternatively, PDIA1 may reduce improper proinsulin disulfide bonds as demonstrated during infection with pathogens that require reduction for retro-translocation from the ER to the cytosol and from evidence that supports a role for PDI as a reductase important for degradation of mutant Akita proinsulin and mutant thyroglobulin.

Proinsulin disulfide maturation in the ER is absolutely required for proinsulin export to the Golgi complex for delivery to immature granules. If PDIA1 assists in disulfide isomerization to facilitate correct disulfide bonding in proinsulin, its absence could increase aberrant disulfide-linked proinsulin complexes (FIG. 20). Although the role of PDIA1 in vivo is complicated by the presence of other ER oxidoreductases and glutathione, based on the greater accumulation of HMW disulfide-linked proinsulin complexes in Pdia1-null islets, the data strongly suggest that PDIA1 participates in the resolution/dissolution of these inappropriate disulfide-linked complexes. This could include both PDI chaperone function as well as oxidoreductase function. It is important to note that healthy murine WT islets also exhibit a lower level of these HMW complexes in addition to smaller oligomeric disulfide-linked proinsulin species. Importantly, the increased BiP expression in the islets bearing Pdia1-deleted β cells suggests that increased accumulation of inappropriate disulfide-linked proinsulin complexes may induce ER stress, supporting a link between aberrant disulfide-linked proinsulin complexes and a compromise in B cell health and function.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for determining a likelihood of developing type II diabetes in a subject, the method comprising:
detecting the presence of an aberrant proinsulin complex in a sample of the subject,
wherein the aberrant proinsulin complex is correlated with an insulin tolerance of the subject.

2. The method of claim 1, wherein the sample comprises a tissue, a tissue culture, a cell, a cell extract, or a bodily fluid.

3. The method of any one of claim 1 or 2, wherein the sample comprises a pancretic islet tissue.

4. The method of any one of claim 1 or 2, wherein the sample comprises a pancreatic beta cell.

5. The method of any one of claims 1 to 4, wherein the aberrant proinsulin complex comprises a misfolded proinsulin peptide.

6. The method of any one of claim 1 or 5, wherein the aberrant proinsulin complex comprises a reactive thiol group. The method of claim 6, wherein the aberrant proinsulin complex comprises at least a dimer of proinsulin peptides.

8. The method of claim 7, wherein the dimer of the proinsulin peptides is formed by a Cys(B19)-Cys(B19) disulfide bond between two proinsulin peptides.

9. The method of any one of claims 1 to 8, wherein the detecting the aberrant proinsulin complex comprises contacting the sample with an antibody that is specific to a proinsulin peptide.

10. The method of claim 9, wherein the antibody is CCI-17.

11. The method of any one of claims 1 to 10, further comprising measuring an amount of the aberrant proinsulin complex.

12. The method of claim 11, further comprising determining a high likelihood of developing type II diabetes when the amount of the aberrant proinsulin complex exceeds a predetermined threshold.

13. The method of claim 12, wherein the predetermined threshold is at least 10% of an amount of mature insulin protein in the sample.

14. The method of claim 12, wherein the predetermined threshold is at least 30% of an amount of mature insulin protein in the sample.

15. The method of claim 12, wherein the predetermined threshold is at least 10% of total proinsulin peptides in the sample.

16. The method of claim 12, wherein the predetermined threshold is at least 30% of total proinsulin peptides in the sample.

17. The method of claim 12, wherein the predetermined threshold is at least 10% more of the aberrant proinsulin complex compared to an amount of the aberrant proinsulin complex detected in a healthy subject.

18. The method of claim 12, wherein the predetermined threshold is at least 30% more of the aberrant proinsulin complex compared to an amount of the aberrant proinsulin complex detected in a healthy subject.

19. A method of determining a likelihood of developing type II diabetes in a subject, the method comprising:
detecting an abnormality of an ER oxidoreductase in a sample of the subject, wherein the abnormality of the ER oxidoreductase is associated with a presence of an aberrant proinsulin complex.

20. The method of claim 19, the sample comprises a tissue, a tissue culture, a cell, a cell extract, or a bodily fluid.

21. The method of any one of claim 19 or 20, wherein the sample comprises a pancreatic islet tissue.

22. The method of any one of claim 19 or 20, wherein the sample comprises a pancreatic beta cell.

23. The method of any one of claims 19 to 22, wherein the aberrant proinsulin complex comprises a misfolded proinsulin peptide.

24. The method of any one of claims 19 to 23, wherein the aberrant proinsulin complex comprises a reactive thiol group.

25. The method of claim 24, wherein the aberrant proinsulin complex comprises at least a dimer of proinsulin peptides.

26. The method of claim 25, wherein the dimer of the proinsulin peptides is formed by a Cys(B19)-Cys(B19) disulfide bond between two proinsulin peptides.

27. The method of any one of claims 19 to 26, wherein the ER oxidoreductase is protein disulfide isomerase A1.

28. The method of any one of claims 19 to 27, wherein the abnormality comprises a mutation, a reduced activity, a reduced expression, or an intracellular mislocalization.

29. The method of any one of claims 19 to 28, further comprising determining a high likelihood of developing type II diabetes when the abnormality of the ER oxidoreductase exceeds a predetermined threshold.

30. The method of claim 29, wherein the predetermined threshold is at least 20% of reduced expression of the ER oxidoreductase.

31. The method of claim 29, wherein the predetermined threshold is at least 20% of reduced activity of the ER oxidoreductase.

32. A method of preventing beta cell dysfunction in a subject in need thereof, the method comprising:
suppressing formation of an aberrant proinsulin complex by facilitating an activity of an ER oxidoreductase.

33. The method of claim 32, wherein the aberrant proinsulin complex comprises at least a dimer of proinsulin peptides.

34. The method of claim 33, wherein the dimer of the proinsulin peptides is formed by a Cys(B19)-Cys(B19) disulfide bond between two proinsulin peptides.

35. The method of any one of claims 32 to 34, wherein the ER oxidoreductase is protein disulfide isomerase A1.

36. The method of any one of claims 32 to 35, wherein the facilitating the activity of the ER oxidoreductase comprises overexpressing the ER oxidoreductase in a pancreatic cell of the subject.

37. The method of any one of claims 32 to 36, wherein the facilitating the activity of the ER oxidoreductase comprises activating a signaling pathway associated with an expression of the ER oxidoreductase.

38. The method of any one of claims 32 to 37, further the facilitating the activity of the ER oxidoreductase is combined with an anti-diabetic therapy.

39. The method of claim 38, wherein the anti-diabetic therapy comprises metformin, acarbose, or combinations thereof.


SEQUENCE LISTING (66 sequences)

SEQ ID NO: 1 - protein, 4 aa, Artificial Sequence
  Description of Artificial Sequence Synthetic peptide
KDEL

SEQ ID NO: 2 - protein, 14 aa, Homo sapiens
PLALEGSLQKRGIV

SEQ ID NO: 3 - protein, 14 aa, Homo sapiens
EAEDLQVGQVELGG

SEQ ID NO: 4 - protein, 12 aa, Homo sapiens
YTPKTRREAEDL

SEQ ID NO: 5 - protein, 9 aa, Rattus sp.
RQKRGIVEQ

SEQ ID NO: 6 - DNA, 24 bp, Artificial Sequence
  Description of Artificial Sequence Synthetic primer
ccagagcgaaagcatttgccaaga

SEQ ID NO: 7 - DNA, 24 bp, Artificial Sequence
  Description of Artificial Sequence Synthetic primer
tcggcatcgtttatggtcggaact

SEQ ID NO: 8 - DNA, 20 bp, Artificial Sequence
  Description of Artificial Sequence Synthetic primer
gtttgacaaaagcctgggtg

SEQ ID NO: 9 - DNA, 23 bp, Artificial Sequence
  Description of Artificial Sequence Synthetic primer
ccagctataatcagagaccatca

SEQ ID NO: 10 - DNA, 20 bp, Artificial Sequence
  Description of Artificial Sequence Synthetic primer
ggtctgaaggtcacctgctc

SEQ ID NO: 11 - DNA, 20 bp, Artificial Sequence
  Description of Artificial Sequence Synthetic primer
ggagcgtggcttcttctaca

SEQ ID NO: 12 - DNA, 21 bp, Artificial Sequence
  Description of Artificial Sequence Synthetic primer
caagatcaagccccacctgat

SEQ ID NO: 13 - DNA, 21 bp, Artificial Sequence
  Description of Artificial Sequence Synthetic primer
agttccccccaaccagtactt

SEQ ID NO: 14 - DNA, 21 bp, Artificial Sequence
  Description of Artificial Sequence Synthetic primer
gatggaattgtcagccacttg

SEQ ID NO: 15 - DNA, 21 bp, Artificial Sequence
  Description of Artificial Sequence Synthetic primer
ggtgtgtgcaaatcggtagtt

SEQ ID NO: 16 - DNA, 20 bp, Artificial Sequence
  Description of Artificial Sequence Synthetic primer
agctccttggcagctttctc

SEQ ID NO: 17 - DNA, 21 bp, Artificial Sequence
  Description of Artificial Sequence Synthetic primer
tgcagacattattttggtgga

SEQ ID NO: 18 - DNA, 21 bp, Artificial Sequence
  Description of Artificial Sequence Synthetic primer
ctagcagtcagcggtctgtat

SEQ ID NO: 19 - DNA, 20 bp, Artificial Sequence
  Description of Artificial Sequence Synthetic primer
cacaggccgtcactctgaat

SEQ ID NO: 20 - DNA, 18 bp, Artificial Sequence
  Description of Artificial Sequence Synthetic primer
ctccatctgcttgtccat

SEQ ID NO: 21 - DNA, 19 bp, Artificial Sequence
  Description of Artificial Sequence Synthetic primer
ggctgcacacactctttac

SEQ ID NO: 22 - DNA, 20 bp, Artificial Sequence
  Description of Artificial Sequence Synthetic primer
acgggtcctcttgttttcct

SEQ ID NO: 23 - DNA, 20 bp, Artificial Sequence
  Description of Artificial Sequence Synthetic primer
tggatgaaatccaccaaagc

SEQ ID NO: 24 - DNA, 20 bp, Artificial Sequence
  Description of Artificial Sequence Synthetic primer
ttctcgctctccagaatgtg

SEQ ID NO: 25 - DNA, 20 bp, Artificial Sequence
  Description of Artificial Sequence Synthetic primer
gaggaggtcatccgactgaa

SEQ ID NO: 26 - DNA, 20 bp, Artificial Sequence
  Description of Artificial Sequence Synthetic primer
ccgaactggaaggaactcag

SEQ ID NO: 27 - DNA, 20 bp, Artificial Sequence
  Description of Artificial Sequence Synthetic primer
ggattaagcggacaattcca

SEQ ID NO: 28 - DNA, 19 bp, Artificial Sequence
  Description of Artificial Sequence Synthetic primer
tgccttgcaccagcattat

SEQ ID NO: 29 - DNA, 20 bp, Artificial Sequence
  Description of Artificial Sequence Synthetic primer
tgtctacacctgttcgcagc

SEQ ID NO: 30 - DNA, 24 bp, Artificial Sequence
  Description of Artificial Sequence Synthetic primer
cctcctacagcagtggtgattaca

SEQ ID NO: 31 - DNA, 21 bp, Artificial Sequence
  Description of Artificial Sequence Synthetic primer
gggtctctgtgcagtcattgt

SEQ ID NO: 32 - DNA, 20 bp, Artificial Sequence
  Description of Artificial Sequence Synthetic primer
agacaatgggaagacggttg

SEQ ID NO: 33 - DNA, 20 bp, Artificial Sequence
  Description of Artificial Sequence Synthetic primer
ttgaagcatagccgtcacag

SEQ ID NO: 34 - DNA, 20 bp, Artificial Sequence
  Description of Artificial Sequence Synthetic primer
tactgctcacgaatacagtt

SEQ ID NO: 35 - DNA, 20 bp, Artificial Sequence
  Description of Artificial Sequence Synthetic primer
gctcaggtaattgaagtctt

SEQ ID NO: 36 - DNA, 20 bp, Artificial Sequence
  Description of Artificial Sequence Synthetic primer
ctgcctttcaccttggagac

SEQ ID NO: 37 - DNA, 20 bp, Artificial Sequence
  Description of Artificial Sequence Synthetic primer
cgtttcctggggatgagata

SEQ ID NO: 38 - DNA, 21 bp, Artificial Sequence
  Description of Artificial Sequence Synthetic primer
ggtgcagcaggacatcaagtt

SEQ ID NO: 39 - DNA, 21 bp, Artificial Sequence
  Description of Artificial Sequence Synthetic primer
cccacctccaatatcaacttg

SEQ ID NO: 40 - DNA, 20 bp, Artificial Sequence
  Description of Artificial Sequence Synthetic primer
aagaacacgcttgggaatgg

SEQ ID NO: 41 - DNA, 18 bp, Artificial Sequence
  Description of Artificial Sequence Synthetic primer
actccccttggcctccac

SEQ ID NO: 42 - DNA, 16 bp, Artificial Sequence
  Description of Artificial Sequence Synthetic primer
gagtccgcagcaggtg

SEQ ID NO: 43 - DNA, 18 bp, Artificial Sequence
  Description of Artificial Sequence Synthetic primer
gtgtcagagtccatggga

SEQ ID NO: 44 - DNA, 21 bp, Artificial Sequence
  Description of Artificial Sequence Synthetic primer
cacaggtacagtcgtccaggt

SEQ ID NO: 45 - DNA, 19 bp, Artificial Sequence
  Description of Artificial Sequence Synthetic primer
cttgctcgttggactcctg

SEQ ID NO: 46 - DNA, 20 bp, Artificial Sequence
  Description of Artificial Sequence Synthetic primer
aatagaaagaatgcttcgcc

SEQ ID NO: 47 - DNA, 20 bp, Artificial Sequence
  Description of Artificial Sequence Synthetic primer
tcttcagggtcttcttctgg

SEQ ID NO: 48 - DNA, 20 bp, Artificial Sequence
  Description of Artificial Sequence Synthetic primer
tactgatggacgtggaaccc

SEQ ID NO: 49 - DNA, 19 bp, Artificial Sequence
  Description of Artificial Sequence Synthetic primer
gaaccatccacttcgagca

SEQ ID NO: 50 - DNA, 20 bp, Artificial Sequence
  Description of Artificial Sequence Synthetic primer
gcttgatagcctccagcaac

SEQ ID NO: 51 - DNA, 20 bp, Artificial Sequence
  Description of Artificial Sequence Synthetic primer
actgaagttcaatggtgggg

SEQ ID NO: 52 - DNA, 20 bp, Artificial Sequence
  Description of Artificial Sequence Synthetic primer
gtttcccgtgcaatcagttc

SEQ ID NO: 53 - DNA, 21 bp, Artificial Sequence
  Description of Artificial Sequence Synthetic primer
caatgtaaaattgggctcgaa

SEQ ID NO: 54 - DNA, 20 bp, Artificial Sequence
  Description of Artificial Sequence Synthetic primer
tcctgctacctcccagaaga

SEQ ID NO: 55 - DNA, 20 bp, Artificial Sequence
  Description of Artificial Sequence Synthetic primer
tgagacctcaaagcagcttc

SEQ ID NO: 56 - DNA, 21 bp, Artificial Sequence
  Description of Artificial Sequence Synthetic primer
aaccacacccacaggatcaga

SEQ ID NO: 57 - DNA, 22 bp, Artificial Sequence
  Description of Artificial Sequence Synthetic primer
tcttcgctttattgctccatga

SEQ ID NO: 58 - DNA, 20 bp, Artificial Sequence
  Description of Artificial Sequence Synthetic primer
gccaccacgctcttctgtct

SEQ ID NO: 59 - DNA, 20 bp, Artificial Sequence
  Description of Artificial Sequence Synthetic primer
gtctgggccatggaactgat

SEQ ID NO: 60 - DNA, 21 bp, Artificial Sequence
  Description of Artificial Sequence Synthetic primer
ataaaaagaggctgtgaacgg

SEQ ID NO: 61 - DNA, 16 bp, Artificial Sequence
  Description of Artificial Sequence Synthetic primer
ggtccaagagagacga

SEQ ID NO: 62 - DNA, 21 bp, Artificial Sequence
  Description of Artificial Sequence Synthetic primer
cgagaacggtggaactttgac

SEQ ID NO: 63 - DNA, 19 bp, Artificial Sequence
  Description of Artificial Sequence Synthetic primer
tcccagacgaagttgccct

SEQ ID NO: 64 - protein, 4 aa, Artificial Sequence
  Description of Artificial Sequence Synthetic peptide
CGHC

SEQ ID NO: 65 - DNA, 12 bp, Artificial Sequence
  Description of Artificial Sequence Synthetic oligonucleotide
tgtggccactgc

SEQ ID NO: 66 - DNA, 12 bp, Artificial Sequence
  Description of Artificial Sequence Synthetic oligonucleotide
tgtggtcactgc